(19) United States
(12) Patent Application Publication
(10) Pub. No.: US 2021/0270852 A1
(43) Pub. Date: Sep. 2, 2021

(54) Methods of Detecting Neuroaxonal Dystrophy Disorders Associated with Vitamin E Deficiency and Uses Thereof

(71) Applicant: The Regents of the University of California, Oakland, CA (US)

(72) Inventors: Carrie Finno, Davis, CA (US); Birgit Puschner, Davis, CA (US)

(21) Appl. No.: 16/972,495

(22) PCT Filed: Jun. 12, 2019
(86) PCT No.: PCT/US2019/036808

§ 371 Date: Dec. 4, 2020

Related U.S. Application Data
(60) Provisional application No. 62/836,515, filed on Apr. 19, 2019.
(60) Provisional application No. 62/685,462, filed on Jun. 15, 2018.

(51) Int. Cl. G01N 33/82

(52) U.S. Cl. CPC G01N 33/82; G01N 2800/28

(57) ABSTRACT

Methods of detecting neuroaxonal dystrophy associated with vitamin E deficiency in a non-human subject are provided. Methods of detecting an equine neuroaxonal dystrophy (eNAD)/equine degenerative myeloencephalopathy (EDM) (eNAD/EDM) disorder in an equine subject, including the presence or the absence of such a disorder, are provided. The subject methods may involve identifying an elevated rate of alpha-tocopherol metabolism in the subject. Methods of treating non-human subjects for neuroaxonal associated with vitamin E deficiency, including eNAD/EDM disorders, are provided as well. Also provided are methods of screening a non-human subject for breeding and/or breeding such non-human subjects, wherein the methods involve detecting the presence or absence of a neuroaxonal dystrophy associated with vitamin E deficiency, including an eNAD/EDM disorder, in the subject. Kits, reagents and/or devices for use in performing the herein described methods are also provided.

### CROSS-REFERENCE TO RELATED APPLICATIONS

Pursuant to 35 U.S.C. § 119 (e), this application claims priority to the filing date of the U.S. Provisional Patent Application Ser. No. 62/685,462, filed Jun. 15, 2018, and Ser. No. 62/836,515, filed Apr. 19, 2019, the disclosures of which are herein incorporated by reference.

### STATEMENT REGARDING FEDERALLY SPONSORED RESEARCH

This invention was made with Government support under Grant No. OD015134, awarded by the National Institutes of Health (NIH). The Government has certain rights in the invention.

### INTRODUCTION

Equine neuroaxonal dystrophy (eNAD) is a neurological disease in which neuron cell bodies and the axons throughout the brain and spinal cord undergo degeneration. Neuroaxonal dystrophies affect various animals including horses, humans, dogs, cats and sheep. However, these diseases can be distinctly different in different species. A genetic basis for certain neuroaxonal dystrophies has been identified in humans and is suspected in sheep, cats and dogs. In horses, eNAD appears to be inherited, as suggested by pedigree analysis of Morgans, Appaloosas, and Quarter Horses and supported by breeding studies in Morgan horses. Adult horses deficient in vitamin E may develop a vitamin E deficient myopathy or equine motor neuron disease (EMND)

Equine NAD is considered the underlying basis of equine degenerative myeloencephalopathy (EDM), a degenerative and irreversible disease of young horses characterized by hypermetria of the limbs. Hypermetria in horses is typified by an overshoot of intended position with the leg, appearing as an inability to judge distance or scale. eNAD and EDM differ primarily in degree and differences between eNAD and EDM appear to depend on the specific areas of the central nervous system affected.

Equine NAD/EDM is the second most prevalent neurological disease in horses, in one study eNAD/EDM represented 24% of all cases of neurological diseases in the overall study population. Males and females are equally affected by the disease.

Horses suffering from eNAD or EDM typically display a symmetric (left to right) incoordination (ataxia) that may be more severe in the hind limbs than in the forelimbs. Clinical signs may appear as early as 6-12 months of life; however, these neurological abnormalities can be subtle and may be missed for years unless the horse is specifically examined for neurological disease. Treatments have some efficacy if applied at or before 2 years of life; however, efficacy is limited after 2 years of age.

Mild cases may present, resulting in performance-related issues, where the horse does not perform up to standard expectations for its breeding and training. Mild cases may be correspondingly difficult to identify in affected individuals.

Conventionally, a definitive diagnosis of eNAD/EDM requires examination of the spinal cord on post-mortem examination, such that without a necropsy (post-mortem examination), these diseases cannot be definitively diagnosed using conventional methods.

### SUMMARY

Methods of detecting neuroaxonal dystrophy associated with vitamin E deficiency in a non-human subject are provided. Methods of detecting an equine neuroaxonal dystrophy (eNAD)/equine degenerative myeloencephalopathy (EDM) (eNAD/EDM) disorder in an equine subject, including the presence or the absence of such a disorder, are provided. The subject methods may involve identifying an elevated rate of alpha-tocopherol metabolism in the subject. Methods of treating non-human subjects for neuroaxonal dystrophy associated with vitamin E deficiency, including eNAD/EDM disorders, are provided as well. Also provided are methods of screening a non-human subject for breeding and/or breeding such non-human subjects, wherein the methods involve detecting the presence or absence of a neuroaxonal dystrophy associated with vitamin E deficiency, including an eNAD/EDM disorder, in the subject. Kits, reagents and/or devices for use in performing the herein described methods are also provided.

### BRIEF DESCRIPTION OF THE FIGURES

FIG. 1 provides a schematic depiction of vitamin E transport as referenced herein.

FIG. 2 depicts a protocol for administration of bioavailable RRR-alpha-tocopherol and sample collection according to an embodiment described herein.

FIG. 3 provides a time course of measured serum α-TOH levels in healthy control and eNAD affected horses.

FIG. 4 provides a time course of measured serum γ-TOH levels in healthy control and eNAD affected horses.

FIG. 5 provides a time course of measured serum α-CMBHC levels in healthy control and eNAD affected horses.

FIG. 6 provides a time course of measured serum α-CEHC levels in healthy control and eNAD affected horses.

FIG. 7 provides a time course of measured serum γ-CEHC levels in healthy control and eNAD affected horses.

### DEFINITIONS

The terms “treatment”, “treating”, “treat” and the like are used herein to generally refer to obtaining a desired pharmacologic and/or physiologic effect. The effect can be prophylactic in terms of completely or partially preventing a disease or symptom(s) (used interchangeably with the term “clinical sign(s)”) thereof and/or may be therapeutic in terms of a partial or complete stabilization or cure for a disease and/or adverse effect attributable to the disease. The term “treatment” encompasses any treatment of a disease in a mammal, particularly a non-human mammal, and includes: (a) preventing the disease and/or symptom(s) from occurring in a non-human subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted (e.g., those with the dysfunction or deficiency) as well as those in which prevention is desired (e.g., those with increased susceptibility to the dysfunction or deficiency; those suspected of having the dysfunction or deficiency; those having one or more risk factors for the dysfunction or deficiency, etc.).

The terms “recipient”, “individual”, “subject”, “host”, and “patient”, are used interchangeably herein and refer to any mammalian subject for whom diagnosis, treatment, or therapy is desired, particularly non-human subjects. “Mammal” for purposes of treatment refers to any animal classified as a mammal, including non-human primates, domestic and farm animals, and zoo, sports, or pet animals, such as dogs, horses, cats, cows, sheep, goats, pigs, camels, etc.

The term “assessing” includes any form of measurement, and includes determining if an element is present or not. The terms “determining”, “measuring”, “evaluating”, “assessing” and “assaying” are used interchangeably and include quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

The terms “Quarter Horse” and “American Quarter Horse” refer to a breed of horse. Breed characteristics include small, short, refined head with a straight profile, and a strong, well-muscled body, featuring a broad chest and powerful, rounded hindquarters. They usually stand between 14 and 16 hands (56 and 64 inches, 142 and 163 cm) high, although some Halter-type and English hunter-type horses may grow as tall as 17 hands (68 inches, 173 cm). There are two main body types: the stock type and the hunter or racing type. The stock horse type is shorter, more compact, stocky and well-muscled, yet agile. The racing and hunter type Quarter Horses are somewhat taller and smoother muscled than the stock type. Horses shown in-hand in Halter competition are larger yet, with a very heavily muscled appearance, while retaining small heads with wide jowls and refined muzzles. Quarter Horses come in nearly all colors. The primary breed registry for American Quarter Horses is with the American Quarter Horse Associate (aqha.com).

The term “Morgan” refers to a breed of horse. Breed characteristics include stamina and vigor, personality and eagerness and strong natural way of moving. The head should be expressive with broad forehead; large prominent eyes; with straight or slightly dished short face; firm fine lips; large nostrils and well-rounded jowls. The ears should be short and shapely, set rather wide apart and carried alertly. Mares may have a slightly longer ear. The throatlatch is slightly deeper than other breeds and should be refined sufficiently to allow proper flexion at the poll and normal respiration. The neck should come out on top of an extremely well-angulated shoulder with depth from top of withers to point of shoulder. It should be relatively fine in relation to sex. It should be slightly arched and should blend with the withers and back. The top line of the neck should be considerably longer than the bottom line. The stallion should have more crest than the mare or gelding. An animal gelded late in life may resemble the stallion more closely. The withers should be well defined and extend into the back in proportion to the angulation of the shoulder. The body should be compact with a short back, close coupling, broad loins, deep flank, well-sprung ribs, croup long and well-muscled with tail attached high, carried gracefully and straight. A weak, low, or long back is a severe fault. The Morgan horse should not be higher at the croup than at the withers. The stifle should be placed well forward and low in the flank area. The legs should be straight and sound with short cannons, flat bone, and an appearance of overall substance with refinement. The forearm should be relatively long in proportion to the cannon. The pasterns should have sufficient length and angulation to provide a light, springy step. The structure of the rear legs is of extreme importance to the selection of a long-lasting equine athlete. Any sign of poor angulation of the hocks, sickle hocks or cow hocks must be considered a severe fault. Lack of proper flexion of the hock is cause for very close examination of the entire structure of the rear legs and should not be tolerated in breeding stock or show ring winners. The feet should be in proportion to the size of the horse, round, open at heel, with concave sole and hoof of dense structure. Viewed from the front, the chest should be well developed. The front legs should be perpendicular to the ground and closely attached to the body. Viewed from the side, the top line represents a gentle curve from the poll to the back, giving the impression of the neck sitting on top of the withers rather than in front of them, continuing to a short, straight back and a relatively level croup rounding into a well-muscled thigh. The tail should be attached high and carried well-arched. At maturity the croup should NOT be higher than the withers. The under line should be long and the body deep through the heart girth and flanks. The extreme angulation of the shoulder results in the arm being a little more vertical than in other breeds, placing the front legs slightly farther forward on the body. The front legs should be straight and perpendicular to the ground. The rear cannons should be perpendicular to the ground when points of hocks and buttocks are in the same vertical lines. Viewed from the rear, the croup should be well rounded, thighs and gaskins well-muscled. Legs should be straight. The gaskin should be relatively long in relation to the cannon. The Morgan should portray good spring of rib and well-rounded buttocks. Slab-sided individuals should be faulted. The height ranges from 14.1 to 15.2 hands, with some individuals under or over.

The terms “Appaloosa” and “Paint” refer to a breed of horse. Breed characteristics include distinctive patterned coat (though not present in all registered individuals), striped hooves, mottled skin, and eyes where you can see white sclera even when the eye is held in its normal position. The coat color of an Appaloosa is a combination of a base color (including e.g., black, grey, chestnut, bay, buckskin, palmino, cremello or perlino, grulla, and dun) and an overlaid spotting pattern. Mottled skin is typically seen around the eyes, muzzle, genitalia and anus. The Appaloosa's spotting patterns are collectively known as the leopard-complex. Any horse that displays Appaloosa core characteristics, such as the distinctive coat patterns, the mottled skin, the striped hooves, and the visible white sclera, is a carrier of at least one allele of the dominant leopard-complex (LP) gene. A horse that is heterozygous for LP is normally darker than a horse that is just homozygous for LP, although there are many exceptions. With the leopard-complex being the primary identifying factor of the breed, a wide range of body types can be seen among the registered Appaloosas. This reflects how many different horse breeds influenced, and to a certain degree continue to influence, the Appaloosa breed. The height varies from 14 to 16 hands. The smallest adult Appaloosas tend to weigh around 950 lbs, while the heaviest can weigh around 1,250 lbs.

The term “Haflinger” refers to a breed of horse. Breed characteristics include great strength relative to its size, handsome appearance and a gentle disposition. One of the prominent and most consistent characteristics of the Haflinger is its color. It is always chestnut, in varying shades, and the mane and tail are consistently flaxen or cream-colored. White markings are acceptable. The head is large with wide-set eyes. The neck is substantial and the mane is, if not clipped, long and full, as is the tail. The body is relatively long and the back is broad; the chest is full. It has powerful quarters and short legs with a limited amount of feather about the fetlocks. It stands at about 14 hands and often between 140 and 155 cm. It is noted for its longevity. The breed origin can be traced to medieval times when writings told of an Oriental breed of horse found in the Southern Tyrolean Mountains of present-day Austria and northern Italy, though the modern Haflinger is now found all over the world.

The term “Standardbred” refers to a breed of horse. Breed characteristics include ability to race in harness at a trot or pace instead of under saddle at a gallop. Developed in North America, the breed is now recognized worldwide for its harness racing ability. They are solid, well-built horses with good dispositions. A Standardbred is a bit heavier in build than a Thoroughbred, but still shows quality and refinement. Standardbreds tend to be more muscled and longer bodied than the American Thoroughbred. They also are of more placid dispositions, as suits horses whose races involve more strategy and more changes of speed than do Thoroughbred races. Standardbreds are considered people-oriented, easy-to-train horses. They are generally a bit heavier in build than their Thoroughbred cousins, but have refined, solid legs and powerful shoulders and hindquarters. Standardbreds have a wide range of height, from 14.1 to 17 hands (57″-66″), and most often are bay or the darker variation of bay called “brown”, although other colors such as chestnut and black are not uncommon, including chestnut, black, gray and roan are also found. The tobiano pattern is seen in some New Zealand-bred horses. There are two basic types, trotters and pacers. As the name suggests, the trotters preferred racing gait is the trot. The pace is a two beat lateral gait; Pacers forelegs move in unison with the hind legs on the same side. However, the breed also is able to perform all other horse gaits, including the canter, and pacers can be retrained to trot.

The terms “Thoroughbred” and “TB” refer to a breed of horse. Breed characteristics include a refined head, long neck, sloping shoulders, deep body, muscular hindquarters and fine long legs. The Thoroughbred horse is spirited and bold. The Thoroughbred horse was developed in England where it was bred for racing and exported across the world. Thoroughbred horses are so inbred that the pedigree of every horse can be traced back to one of three stallions, Byerley Turk (1680-1696), Darley Arabian (1700-1733) and the Godolphin Arabian (1724-1753), and these are known as the “Foundation sires”. The Thoroughbred horse is any solid color. Thoroughbred horses may have white face markings and/or white leg markings. The Thoroughbred horse stands 14.2 to 17.2 hands, averaging 16 hands (64 inches, or 163 cm) high and weighing about 1,000 pounds (450 kg) at maturity. The Thoroughbred is often used as a racehorse, riding horse and competition horse. A horse having only one Thoroughbred parent is called a Grade Thoroughbred in the United States and a half-bred in Great Britain.

The term “pony” refers to a small horse, which depending on context may be a horse that is under an approximate or exact height at the withers or a small horse with a specific conformation and temperament. Ponies are often grouped into small, medium, and large sizes. Small ponies are 12.2 hands (50 inches (130 cm)) and under, medium ponies are over 12.2 but no taller than 13.2 hands (54 inches (140 cm)), and large ponies are over 13.2 hands but no taller than 14.2 hands. Compared to other horses, ponies often exhibit thicker manes, tails and overall coat, as well as proportionally shorter legs, wider barrels, heavier bone, thicker necks, and shorter heads with broader foreheads. Individual pony breeds include but are not limited to e.g., American Shetland, American Walking Pony, Anadolu pony, Ariegeois Pony, Assateague Pony, Asturian pony, Australian Pony, Bali Pony, Bashkir Pony, Basque Pony, Basuto pony, Batak Pony, Bhutia Pony, Bosnian Pony, British Riding Pony, British Spotted Pony, Burmese Pony, Carpathian Pony, Canadian rustic pony, Caspian pony, Chincoteague Pony, Chinese Guoxia, Coffin Bay Pony, Connemara pony, Czechoslovakian Small Riding Pony, Dales Pony, Danish Sport Pony, Dartmoor pony, Deli pony, Deutsches Reitpony, Dulmen Pony, Eriskay pony, Esperia Pony, Exmoor pony, Falabella, Faroe pony, Fell Pony, Flores pony, French Saddle Pony, Galician Pony, Garrano, Gayoe, German Riding Pony, German Classic Pony, Gotland Pony, Guizhou pony, Guangxi, Guo-xia pony, Hackney pony, Highland Pony, Hokkaido Pony, Hucul Pony, Hunter Pony, Icelandic pony, Indian Country Bred, Java Pony, Kerry bog pony, Landais Pony, Lijiang pony, Lundy Pony, Manipuri Pony, Miyako Pony, Narym Pony, New Forest pony, Newfoundland pony, Noma pony, Northlands Pony, Ob pony, Peneia Pony, Petiso Argentino, Pindos Pony, Poney Mousseye, Pony of the Americas, Quarter pony, Riding Pony, Sable Island Pony, Sandalwood Pony, Sardinian Pony, Shetland pony, Skogsruss, Skyros Pony, Spiti Pony, Sumba and Sumbawa Pony, Tibetan Pony, Timor Pony, Tokara Pony, Virginia highlander, Vyatka horse, Welara, Welsh pony, Welsh mountain pony, Western Sudan pony, Yakut Pony, Yonaguni, Zaniskari, and Zemaitukas.

The terms “Pony of the Americas” and “POA” refer to a breed of horse. Breed characteristics include distinctive coat patterns, mottled skin, white sclera and striped hooves. Coat patterns vary widely and, over time, some ponies develop additional color. One of the most common colorations is a blanket pattern, which is characterized by white over the loin and hips with dark, round, g-shaped spots. These spots may vary in size from tiny specks to spots four or more inches in diameter. Others will show white over the hips without dark spots. This variation on the blanket pattern is known as snow-capped. Ponies that have white hairs mixed in with the base coat color are said to be roan. Roan POAs often show varnish marks which are darker areas appearing most often on the upper legs, point of the hip, bridge of the nose, and on the cheek bones. These dark patches have smooth edges that gradually blend into the hair in the surrounding area. Mottled or parti-colored skin is unique to the Appaloosa and POA. Because of this, it is a decisive indicator of a POA. Different from commonly found pink skin (as found under blazes and stockings) mottled skin is a speckled or blotchy pattern of pigmented and non-pigmented skin. There are several places on a pony where mottled skin can be seen easily. These are the eyes, muzzle, udder or sheath and anus or vulva. White sclera on a POA is usually very visible. All horses and ponies have sclera; it is the area of the eye which encircles the iris (the colored or pigmented portion). The POA's sclera is white and usually readily visible, even when the head is held in a normal, relaxed position. Bold, clearly defined vertically light or dark stripes on the hooves are another POA characteristic; however, it is possible a POA will not exhibit any striping on its hooves.

The terms “Lusitano” and “Andalusian” refer to a breed of horse and, in some instances, two breeds of horse. Breed characteristics include noble, generous, ardent, gentle, and able to endure long suffering. The breed is generally middleweight (around 500 Kgs) with a sub-convex profile throughout the body showing rounded outlines. Height is also medium, measured at the withers at 6 years of age, average height for females is about 15.1 hands (1.55 m) and males about 15.3 hands (1.60 m). The coat is most frequently grey or bay. The head is well proportioned, of medium length, narrow and dry, with the lower jaw not too pronounced and the cheek tending to be long. Slightly sub-convex profile with the forehead in advance of the bones of the eyebrows: the eyes tend to be elliptical in shape (almond shape), big and alive, expressive and confident. The ears are of medium length, fine, narrow and expressive. Neck is of medium length, arched with a narrow hairline: the junction between head and neck is narrow or fine: the neck is deep in the base and well inserted between the shoulders, rising up from the withers without any marked depression. The withers are well defined and long, with a smooth transition from the back to the neck, often higher than the croup. The chest is of medium size, deep and muscular. The ribcage is well developed, long and deep with the ribs obliquely arched into the joint with the column which promotes a short and full flank. The shoulders are long, oblique and well-muscled. The back is well defined and tending towards the horizontal making a smooth union between the withers and loins. The loins are short, wide, muscular, slightly convex, well connected with the back and croup with which they form a continuous harmonious line. The forelegs are well muscled and harmoniously inclined. The upper arm straight and muscular. The cannons slightly long and muscular. The fetlocks are dry, relatively big and with very little hair. The pasterns are relatively long and sloping. The hooves are of good constitution, well defined and proportioned without being too open; the line of the coronet is not very evident. The buttock is short and convex. The thigh is muscular and tends to be short, and is orientated in such a way that the patella or gaskin is in the same vertical line of the hip bone, or point of the hip. The leg is slightly long positioning the hock in the same vertical line of the point of the buttock. The hocks are large, strong and dry. The legs present relatively closed angles. Genetically, both Lusitano and Andalusian breeds are quite similar, however the two have some differences. Andalusians are also known for their distinctive gaits and Lusitanos often have a more fiery temperament than Andalusians. The Lusitano also tends to stand a little taller than the Andalusian. The Andalusian tends to range from 15 to 16 hands, while Lusitanos tend to range from 15.2 to 16.2 hands. Since Lusitanos also tend to have more diverse colors than Andalusians. While both breeds accept a variety of colors, Andalusians are usually gray, while Lusitanos can be bay, palomino, dun, and more.

The term “Paso Fino” refers to a breed of horse. Breed characteristics include a smooth, natural gait that is unique to the breed, showing movement that is balanced and in-sync. Generally, sized from 13 to 15.2 hands with 13.3 to 14.2 being the most typical size. Weight is generally 700 to 1000 pounds. Full size may not be attained until the fifth year. Widely varied in color, with or without markings. The Paso Fino is an extremely willing horse that truly seems to enjoy human companionship and strives to please. It is spirited and responsive under tack while sensible and gentle at hand. Long and full mane, tail and forelock. Head is well-shaped with an alert and intelligent face. The head is refined and in proportion to the body, with a defined, but not extreme jaw, and large, expressive eyes. The neck is gracefully arched, medium in length, and allowing for a high carriage. Shoulders slope into the withers with great depth through the hearth. The top line should be proportionately shorter than the underline. The back is strong and muscled. The croup is slightly sloping with rounded loins, broad hips, and strong hocks. The tail is carried gracefully when in motion. The legs are straight with refined bones, strong, well-defined tendons, and broad, long forearms with shorter cannons. The thigh and gaskin are strong and muscled but not exaggerated. Pasterns are sloping and medium in length.

The term “Arabian” refers to a breed of horse. Breed characteristics include finely chiseled head, dished face, long arching neck and high tail carriage. In general, Arabians have a short, straight back (usually one less vertebra than is common with other breeds), perfect balance and symmetry, a deep chest, well-sprung ribs, strong legs of thick density and a more horizontal pelvic bone position. Comparatively small head, profile of head straight or preferably slightly concave below the eyes; small muzzle, large nostrils, extended when in action; large, round, expressive, dark eyes set well apart; comparatively short distance between eye and muzzle; deep jowls, wide between the branches; small ears (smaller in stallions than mares), thin and well-shaped, tips curved slightly inward. Neck is long and arched, set on high and running well back into moderately high withers. Back is short. Croup is comparatively horizontal. Tail is in a natural high tail carriage. Viewed from rear, tail should be carried straight. The average Arabian stand 15 hands at the withers and weighs 1,000 pounds.

The terms “Tennessee Walking Horse” and “TWH” refers to a breed of horse. Breed characteristics include a definitive head with small, well placed ears, a long sloping shoulder, a long sloping hip, a fairly short back and short and strong coupling. The bottom line is longer than the top line, allowing for a long stride. The body is substantial, with long clean legs. It's acceptable for the hind legs to be slightly cow-hocked or sickle-hocked. Tennessee Walking Horses come in many different coat colors and patterns. Backs, browns, bays, and chestnuts are common as are buckskins, duns, roans, pintos and palominos. the Tennessee Walking Horse is a composition of Narragansett and Canadian Pacer, Standardbred, Thoroughbred, Morgan, and American Saddlebred stock. The Tennessee Walking Horse performs three distinct gaits: the flat foot walk, running walk and canter. These three are the gaits for which the Tennessee Walking Horse is famous, with the running walk being an inherited, natural gait unique to this breed. Many Tennessee Walking Horses are able to perform the rack, stepping-pace, fox-trot, single-foot and other variations of the famous running walk. Tennessee Walking Horses generally range from 14.3 to 17 hands and weigh 900 to 1200 pounds.

The terms “Norwegian Fjord” and “Fjord” refers to a breed of horse. Breed characteristics include distinctive colorings, mane and head and neck shape. Approximately 90% of all Fjord Horses are brown dun in color and the other 10% are either red dun, gray, white or “uls” dun, or yellow dun. The Fjord Horse retains the “wild” dun color of the original horse as well as the primitive markings which include zebra stripes on the legs and a dorsal stripe that runs from the forelock down the neck and back and into the tail. Dark stripes may also be seen over the withers. Red duns have reddish-brown stripes and body markings. Gray duns have black or very dark gray stripes and markings. The white or “uls” dun is a very light body color with black or gray stripe and markings. The yellow dun have a darker yellow stripe and markings, they may have a completely white forelock, mane and tail. The yellow dun is a very rare color in the breed. The center hair of the mane is dark (usually black) while the outer hair is white. The mane is often cut short so it will stand erect. It is trimmed in a characteristic crescent shape to emphasize the graceful curve of the neck. The white outer hair is then trimmed slightly shorter than the dark inner hair to display the dramatic dark stripe. The head and neck should present an appearance of elegance without coarseness. The head is medium sized and well defined with a broad, flat forehead and a straight or slightly dished face. The eyes are large. Ears are small and alert. The neck of the Fjord is well muscled and crested. The body is short coupled with good depth, large heart girth, and well-developed muscles. The legs are powerful, with substantial bone and excellent feet which are black in color. Fjords generally range in size from 13.2 to 14.2 Hands and weigh between 900 and 1200 pounds at maturity, with a few individuals ranging outside these measurements.

The term “mixed breed” refers to various breeds of horse. A mixed-breed animal is defined as having undocumented or unknown parentage, while a crossbreed generally has known, usually purebred parents of two distinct breeds or varieties. Accordingly, with respect to inherited conditions, a mixed breed may have a known genetic heritage (e.g., known parents of known heritage, such as a known crossbreed) or an unknown genetic heritage (e.g., unknown parents or known parents of unknown heritage).

The term “Vitamin E” collectively refers to a group of eight potent and lipophilic vitamin E isoforms; alpha-, beta-, gamma-, and delta-tocopherol (α-TP (also referred to as α-TOH), β-TP (also referred to as β-TOH), γ-TP (also referred to as γ-TOH), and δ-TP (also referred to as δ-TOH), respectively) as well as alpha-, beta-, gamma-, and delta-tocotrienol (α-TT (also referred to as α-TOT), β-TT (also referred to as β-TOT), γ-TT (also referred to as γ-TOT), and δ-TT (also referred to as δ-TOT), respectively). α-TP has eight stereoisomers, including RRR, SRR, RRS, RSS, RSR, SSR, RSS and SSS. Tocopherols are a group of fat-soluble phenolic compounds having a chromanol ring and a hydrophobic side chain, often 16-carbon phytyl group. Of all vitamin E isoforms, α-TT is generally considered to be the classic vitamin E as it is the major form of tocopherols found in blood and tissues.

Cytochrome P450 mediated w-hydroxylation of tocopherol, followed by β-oxidation of the side chain results in formation of vitamin E metabolites, including carboxyethyl-hydroxychromans (CEHCs), represented by the general structure (I):

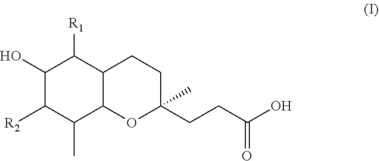

With reference to structure (I), vitamin E metabolites of interest include but are not limited to:

| Compound | R₁ | R₂ | Mol. Weight (g/mol) |
|---|---|---|---|
| α-CEHC | CH3 | CH3 | 278.4 |
| β-CEHC | CH3 | H | 264.3 |
| γ-CEHC | H | CH3 | 264.3 |
| δ-CEHC | H | H | 260.3 |

Important corresponding metabolites formed from the metabolism of vitamin E isoforms include alpha-carboxyethyl-hydroxychroman (α-CEHC), alpha-5-(6-hydroxy-2,5,7,8-tetramethyl-chroman-2-yl)-2-methyl-pentanoic acid (α-CMBHC), and gamma-carboxyethyl-hydroxychroman (γ-CEHC). γ-CEHC is the major metabolite formed from γ-TP metabolism, α-CEHC is the major metabolite formed from α-TP metabolism, and alpha-carboxymethylbutyl hydroxychroman (alpha-CMBHC or α-CMBHC) is a minor vitamin E metabolite formed from α-TP and is a precursor to α-CEHC. After hydroxylation, the metabolites are further subject to Phase II metabolism including glucuronidation and sulfation leading to increased water solubility and enhanced urinary elimination.

The terms “alpha-carboxymethylbutyl hydroxychroman”, “5′-carboxy-alpha-tocopherol”, “5′-Carboxy-alpha-tocopherol”, “2-(4-Carboxy-4-methylbutyl)-6-hydroxy-2,5,7,8-tetramethylchroman”, “3,4-dihydro-6-hydroxy-α,2,5,7,8-pentamethyl-2H-1-benzopyran-2-pentanoic acid”, “alpha-CMBHC” and “α-CMBHC” refer to a metabolite of α-TP. Alpha-CMBHC is a minor α-TP catabolism metabolite, a precursor to α-CEHC, and is a dehydrogenation carboxylate product of 5′-hydroxy-α-tocopherol.

The tocopherols (α-tocopherol, β-tocopherol, γ-tocopherol and δ-tocopherol) and their corresponding tocotrienols are synthesized by plants and have vitamin E antioxidant activity. As described above, they differ in the number and location of methyl groups on the chromanol ring. The naturally occurring form of α-tocopherol is (2R,4′R,8′R)-α-tocopherol (i.e., (R,R,R)-α-tocopherol). Synthetic α-tocopherols are a racemic mixture of eight different R and S stereoisomers. Generally, the 2R forms are recognized as meeting human requirements. The in vivo function of vitamin E is generally considered to scavenge peroxyl radicals via its phenolic (chromanol) hydroxyl group, thus protecting lipids against free radical-catalyzed peroxidation. The tocopheryl radical formed can then be reduced by reductants such as L-ascorbate. Metabolites of α-tocopherol are produced in significant amounts in response to excess vitamin E ingestion. Vitamin E is fat-soluble and its utilization requires intestinal fat absorption mechanisms. It is secreted from the intestine into the lymphatic system in chylomicrons which subsequently enter the plasma. Lipolysis of these chylomicrons can result in delivery of vitamin E to tissues, transfer to high-density lipoproteins (and subsequently to other lipoproteins via the phospholipid exchange protein), or retention in chylomicron remnants. These remnants are taken up by the liver. Natural (R,R,R)-α-tocopherol and synthetic 2R-α-tocopherols are then preferentially secreted from the liver into plasma as a result of the specificity of the α-tocopherol transfer protein. This protein, along with the metabolism of excess vitamin E in the liver and excretion into urine and bile, mediate the supply of α-tocopherol in plasma and tissues.

As used herein, the term metabolite refers to a substance produced during a bodily chemical or physical process. The term “metabolite” includes any chemical or biochemical product of a metabolic process, such as any compound produced by the processing, cleavage or consumption of a biological molecule. Metabolites can be detected in a variety of ways, including assays based on chromatography and/or mass spectrometry, fluorimetry, electrophoresis, immune-affinity, hybridization, immunochemistry, ultra-violet spectroscopy (UV), fluorescence analysis, radiochemical analysis, near-infrared spectroscopy (nearIR or NIRS), nuclear magnetic resonance spectroscopy (NMR), light scattering analysis (LS), and nephelometry.

Metabolites may be analyzed by various methods, including e.g., liquid or gas chromatography or ion mobility (electrophoresis) alone or coupled with mass spectrometry or by mass spectrometry alone. Such methods have been used to identify and quantify biomolecules, such as cellular metabolites. Mass spectrometry methods may be based on, for example, quadrupole, ion-trap, or time-of-flight mass spectrometry, with single, double, or triple mass-to-charge scanning and/or filtering (MS, MS/MS, or MS³) and preceded by appropriate ionization methods such as electrospray ionization, atmospheric pressure chemical ionization, atmospheric pressure photo ionization, matrix-assisted laser desorption ionization (MALDI), or surface-enhanced laser desorption ionization (SELDI). In some embodiments, the first separation of metabolites from a biological sample may be achieved using liquid chromatography. Useful mass spectrometry instruments include quadrupole, ion-trap, or time-of-flight, and Fourier transform instruments among others.

### DETAILED DESCRIPTION

Methods of detecting neuroaxonal dystrophy associated with vitamin E deficiency in a non-human subject are provided. Methods of detecting an equine neuroaxonal dystrophy (eNAD)/equine degenerative myeloencephalopathy (EDM) (eNAD/EDM) disorder in an equine subject, including the presence or the absence of such a disorder, are provided. The subject methods may involve identifying an elevated rate of alpha-tocopherol metabolism in the subject. Methods of treating non-human subjects for neuroaxonal dystrophy associated with vitamin E deficiency, including eNAD/EDM disorders, are provided as well. Also provided are methods of screening a non-human subject for breeding and/or breeding such non-human subjects, wherein the methods involve detecting the presence or absence of a neuroaxonal dystrophy associated with vitamin E deficiency, including an eNAD/EDM disorder, in the subject. Kits, reagents and/or devices for use in performing the herein described methods are also provided.

As summarized above, the present disclosure provides methods of detecting an equine neuroaxonal dystrophy (eNAD)/equine degenerative myeloencephalopathy (EDM) (eNAD/EDM) disorder in an equine subject, including the presence or the absence of such a disorder.

Aspects of the methods may include identifying an elevated rate of alpha-tocopherol metabolism in the subject. Aspects of the methods may also include administering a bioavailable alpha-tocopherol to a subject and measuring a post-administration alpha-carboxymethylbutylhydroxychroman (alpha-CMBHC) concentration in a sample from the subject at one or more timepoints following the administration. Methods of treating non-human subjects for a neuroaxonal dystrophy associated with vitamin E deficiency (such as e.g., an eNAD/EDM disorder) are provided as well, along with methods of screening a non-human subject for breeding and/or breeding such non-human subjects, wherein the methods involve detecting the presence and/or absence of a neuroaxonal dystrophy associated with vitamin E deficiency (such as e.g., an eNAD/EDM) disorder in the subject. Kits, reagents and/or devices for use in performing the herein described methods are also provided.

Before the methods of the present disclosure are described in greater detail, it is to be understood that the methods are not limited to particular embodiments described, as such may, of course, vary. It is also to be understood that the terminology used herein is for the purpose of describing particular embodiments only, and is not intended to be limiting, since the scope of the methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods. The upper and lower limits of these smaller ranges may independently be included in the smaller ranges and are also encompassed within the methods, subject to any specifically excluded limit in the stated range. Where the stated range includes one or both of the limits, ranges excluding either or both of those included limits are also included in the methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods belong. Although any methods similar or equivalent to those described herein can also be used in the practice or testing of the methods, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present methods are not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, which are, for clarity, described in the context of separate embodiments, may also be provided in combination in a single embodiment. Conversely, various features of the methods, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed, to the extent that such combinations embrace operable processes and/or devices/systems/kits. In addition, all sub-combinations listed in the embodiments describing such variables are also specifically embraced by the present methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order which is logically possible.

### Methods

As summarized above, methods are provided for detecting neuroaxonal dystrophy associated with vitamin E deficiency in a non-human subject, including an equine neuroaxonal dystrophy (eNAD)/degenerative myeloencephalopathy (EDM) (eNAD/EDM) disorder in an equine subject. The subject methods will generally involve assessing the metabolism of vitamin E, including assessing one or more vitamin E metabolites, in the subject where an increased rate of metabolism may be indicative of the presence of a neuroaxonal dystrophy associated with vitamin E. In some instances, the subject methods will generally involve assessing the metabolism of alpha-tocopherol, including assessing one or more alpha-tocopherol metabolites, in the subject where an increased rate of metabolism may be indicative of the presence of an eNAD/EDM disorder.

In some embodiments, a subject may be administered a bioavailable alpha-tocopherol and a level of one or more alpha-tocopherol metabolites may be assessed at one or more timepoints following the administration. For example, in some instances, bioavailable alpha-tocopherol may be administered and a post-administration alpha-carboxymethylbutyl hydroxychroman (alpha-CMBHC) concentration may be measured in a sample obtained from the subject.

Methods employing the administration of a bioavailable alpha-tocopherol for assessment of bioavailable alpha-tocopherol metabolism may involve the administration of bioavailable alpha-tocopherol together with other vitamin E isoforms, e.g., other tocopherols and/or tocotrienols, or the administration of alpha-tocopherol alone, i.e., as the sole vitamin E isoform administered. Administered alpha-tocopherol may be natural or synthetic (sometimes referred to as “d-alpha-tocopherol” as compared to “dl-alpha-tocopherol”, respectively). In some instances, natural alpha-tocopherol may be employed for increased bioavailability as compared to synthetic or an increased amount of synthetic (e.g., 2-3 times the amount of natural) may be administered. Administered alpha-tocopherol may include a single stereoisomer (e.g., RRR-α-tocopherol) or multiple (including e.g., two or more, three or more, four or more, five or more, six or more, seven or more, or eight) different stereoisomers. In some instances, administration of a bioavailable alpha-tocopherol in this context may be performed orally. In some instances, administration of a bioavailable alpha-tocopherol in this context may be injected.

Methods employing the administration of vitamin E, e.g., for treatment of a vitamin E deficiency of associated disorder (including e.g., those detected by employing the methods of the present disclosure) may involve the administration of bioavailable alpha-tocopherol together with other vitamin E isoforms, e.g., other tocopherols and/or tocotrienols, or the administration of alpha-tocopherol alone, i.e., as the sole vitamin E isoform administered. For example, for such purposes in some instances, a supplement containing a specific vitamin E isoform (e.g., alpha-tocopherol) may be administered or a supplement or foodstuff containing a variety of isoforms, including two or more, three or more, four or more, five or more, six or more, seven or more, or eight isoforms selected from α-TOH, β-TOH, γ-TOH, δ-TOH, α-TOT, β-TOT, γ-TOT, and δ-TOT, may be employed. In some instances, vitamin E may be administered together with other non-vitamin E agents, including but not limited to e.g., other vitamins such as but not limited to e.g., vitamin A, vitamin D, and the like. In some instances, vitamin E may be administered as the sole active agent, including e.g., where the administration does not include other active agents, such as but not limited to e.g., other vitamins. Such administrations, i.e., where vitamin E is the sole active agent, does not exclude non-active agents, such as but not limited to e.g., pharmaceutically acceptable carriers, additives, solvents, fillers, buffering agents, moistening agents, preservatives, flavoring agents, and the like.

In some instances, vitamin E may be supplied through food and dietary supplements. Abundant sources of vitamin E, and α-TP, include but are not limited to e.g., wheat germ oil, sunflower, canola/rapeseed oil, maize/corn, palm oil, soybean, and olive oil. Other sources of vitamin E include fish, peanut butter, and vegetables (such as e.g., green leafy vegetables). Vegetables generally contain α-TP in highest concentrations in green photosynthesizing portions of the plant. In addition to, or in place of, commonly consumed foods, vitamin E can also be consumed in the form of supplements. Commercial Vitamin E supplements may in some instances, contain only α-TP, including e.g., RRR-α-TP or all-rac-α-TP, often either unesterified or as the ester of acetate, succinate, or nicotinate. In some instances, a vitamin E supplement or vitamin E fortified foodstuff may be formulated together with other non-vitamin E agents, including but not limited to e.g., other vitamins such as but not limited to e.g., vitamin A, vitamin D, and the like. In some instances, a vitamin E supplement or vitamin E fortified foodstuff may include vitamin E as the sole active agent, including e.g., where the formulation does not include other active agents, such as but not limited to e.g., other vitamins. Such formulations, i.e., where vitamin E is the sole active agent, does not exclude non-active agents, such as but not limited to e.g., pharmaceutically acceptable carriers, additives, solvents, fillers, buffering agents, moistening agents, preservatives, flavoring agents, and the like.

Supplements may be supplied in various forms including e.g., powers and liquids. Supplements may be formulated for various uses including but not limited to e.g., formulated for mixture dry feeds, formulated for top-dress on dry feeds, formulated for mixture with drinking water, combinations thereof and the like. In some instances, vitamin E supplements may be formulated for injection and/or intravenous delivery. Vitamin E supplements may be added immediately before (i.e., at the time of) use (e.g., feeding) or within a short time (e.g., within minutes, hours (e.g., within 6 hours, within 12 hours, within 18 hours, etc.), within a day, etc.) before use.

Useful commercially available supplements and feeds containing vitamin E, which may in all or some instances include a bioavailable alpha-tocopherol, include but are not limited to e.g., EMCELLE® TOCOPHEROL (STUARTPRODUCTS, Inc.; Texas, USA), EMCELLE® E-D₃ (STUARTPRODUCTS, Inc.; Texas, USA), MILKADE™ (STUARTPRODUCTS, Inc.; Texas, USA), VITAL E®-500 (STUARTPRODUCTS, Inc.; Texas, USA), VITAL E®-Newborn (STUARTPRODUCTS, Inc.; Texas, USA), VITAL E®-Repro (STUARTPRODUCTS, Inc.; Texas, USA), VITAL E®-A+D (STUARTPRODUCTS, Inc.; Texas, USA), Nano-E® (Kentucky Equine Research; KY, USA), E●CLIPSE® (Kentucky Equine Research; KY, USA), RE-LEVE® (Kentucky Equine Research; KY, USA), RE●LEVE® Sport (Kentucky Equine Research; KY, USA), ALL-PHASE® (Kentucky Equine Research; KY, USA), Elevate® (Kentucky Performance Products; KY, USA), Elevate® SE (Kentucky Performance Products; KY, USA), Elevate® W.S. (Kentucky Performance Products; KY, USA), UltraCruz Equine Natural Vitamin E® Supplement (Santa Cruz Animal Health; TX, USA), UltraCruz Equine Natural Vitamin E® Plus Supplement (Santa Cruz Animal Health; TX, USA), and the like.

As summarized above, methods of the present disclosure may include assessing one or more alpha-tocopherol metabolites in a sample from a non-human subject to determine the presence or absence of neuroaxonal dystrophy associated with vitamin E deficiency in the non-human subject. In some instances, a single alpha-tocopherol metabolite may be assessed. In some instances, the level of an alpha-tocopherol metabolite may be measured, producing a value indicating the level, including absolute or relative level, of the alpha-tocopherol metabolite in the sample. In some instances, assessing the level of an alpha-tocopherol metabolite may include assessing the ratio of the level an alpha-tocopherol metabolite to the level of an alpha-tocopherol. In some instances, the levels of multiple alpha-tocopherol metabolites, including a panel of alpha-tocopherol metabolites, may be assessed. In some instances, the levels of multiple alpha-tocopherol metabolites may be measured, producing multiple values indicating the levels, including absolute or relative levels, of the alpha-tocopherol metabolites in the sample. In some instances, assessing the levels of multiple alpha-tocopherol metabolites may include assessing the ratio of the levels of two or more alpha-tocopherol metabolites to the level of one or more, e.g., two or more, tocopherols and/or one or more, e.g., two or more, tocotrienols.

As summarized above, in some instances, the level of one or more tocopherols and/or tocotrienols. including e.g., alpha-tocopherol and/or alpha-tocotrienol, may be assessed in addition to assessing the level of one or more alpha-tocopherol metabolites. In some instances, assessing the level of one or more tocopherols and/or tocotrienols in addition to tocopherol metabolites may provide for a ratio of tocopherol metabolites to tocopherol/tocotrienol levels, including e.g., alpha tocopherol/tocotrienol levels. In some instances, the level of one or more one or more tocopherols and/or tocotrienols, including all and/or specific tocopherols and/or tocotrienols, including e.g., alpha-tocopherol, may not be assessed and only the level of one or more alpha-tocopherol metabolites may be assessed.

As summarized above, in some instances, a ratio, or one or more ratios, of metabolite to tocopherol and/or tocotrienol level may be assessed in measuring the metabolism of vitamin E of a subject. Any of the herein described metabolites and/or tocopherols and/or tocotrienols may be employed in a subject ratio. Useful ratios include e.g., the ratio of α-CMBHC metabolite level to the level of one or more tocopherol and/or tocotrienol levels, including e.g., alpha-tocopherol and/or alpha-tocotrienol levels. In some instances, a useful ratio may further include the level of α-CEHC. For example, in some embodiments a ratio of the levels of α-CMBHC and α-CEHC to the levels of α-TOH and α-TOT may be employed. In some instances, such a ratio may be referred to as an “α-Ratio” or an “α-Isoform Ratio” and may be mathematically expressed by the following formula: α-Ratio=(α-CMBHC+α-CEHC)/(α-TOH+α-TOT). Useful ratios are not limited to the α-Ratio as described and may include variations where, e.g., an alternative ratio is computed using the same metabolites and tocopherols/tocotrienols in a different mathematical expression and/or certain alternative metabolites are added or substituted in the same or a different mathematical expression.

In some instances, a ratio for assessing metabolism of vitamin E may be produced from the measurement of a panel of analytes, including e.g., where the panel includes multiple metabolites and/or one or more tocopherols/tocotrienols. Useful ratios may, e.g., be derived from a panel including but not limited to a plurality of analytes selected from alpha-CMBHC, alpha-tocopherol (α-TOH), gamma-tocopherol (γ-TOH), alpha-tocotrienol (α-TOT), gamma-tocotrienol (γ-TOT), a pha-carboxyethylhydroxychroman (α-CEHC), and gamma-carboxyethylhydroxychroman (γ-CEHC).

Such assessed levels may be employed to determine whether the non-human subject from which the sample was derived has a neuroaxonal dystrophy associated with vitamin E deficiency. Neuroaxonal dystrophy associated with vitamin E deficiencies may vary and will generally include any neuroaxonal dystrophy, e.g., in an adult, juvenile, newborn, or neonate, that is functionally associated with a vitamin E deficiency. Vitamin E deficiencies resulting in neuroaxonal dystrophy may be inherited, dietary, or a combination thereof.

Determining whether the vitamin E deficiency is present or absent in the non-human subject may be based on whether the detected level of one or more alpha-tocopherol metabolites is above or below, respectively, one or more pre-determined thresholds. Put another way, the presence of the neuroaxonal dystrophy associated with vitamin E deficiency may be detected when the level of the one or more alpha-tocopherol metabolites is above a threshold level, including e.g., where the threshold level is based on the level expected of a healthy subject receiving a normative level of dietary vitamin E. Correspondingly, the absence of the neuroaxonal dystrophy associated with vitamin E deficiency may be detected when the level of the one or more alpha-tocopherol metabolites is at or below a threshold level, including e.g., where the threshold level is based on the level expected of a healthy subject receiving a normative level of dietary vitamin E.

For example, in instances where the amount (i.e., concentration, level, etc.) of a particular metabolite (e.g., alpha-CMBHC) is employed in the assay, the presence or absence of eNAD/EDM may be determined based on whether the assessed amount of the particular metabolite (e.g., alpha-CM BHC) is above or below a particular (e.g., predetermined) threshold, including where such a threshold is an absolute level or a relative level. For example, in some instances a threshold concentration of 9 ng/mL alpha-CMBHC may be employed where e.g., a concentration of at least 9 ng/mL indicates the presence of the eNAD/EDM disorder and a concentration of less than 9 ng/mL indicates the absence of the eNAD/EDM disorder. Useful thresholds may vary, in some instances, and may include but are not limited to e.g., 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, etc.

In some instances, relative thresholds may be employed. For example, in some instances, a measured amount of a metabolite may be compared to a baseline level to arrive at a value for which the metabolite has increased. Useful relative threshold levels include but are not limited to e.g., an at least 1.5-fold increase, an at least 2-fold increase, an at least 3-fold increase, an at least 4-fold increase, an at least 5-fold increase, an at least 6-fold increase, an at least 7-fold increase, an at least 8-fold increase, an at least 9-fold increase, an at least 10-fold increase, or more.

For example, in some embodiments, a threshold of at least a 9-fold increase in alpha-CMBHC may be employed where e.g., at least a 9-fold increase as compared to the baseline alpha-CMBHC indicates the presence of the eNAD/EDM disorder and less than a 9-fold increase as compared to the baseline alpha-CMBHC indicates the absence of the eNAD/EDM disorder. Useful thresholds may vary, in some instances, and may include but are not limited to e.g., a 1.5-fold increase, a 2-fold increase, a 3-fold increase, a 4-fold increase, a 5-fold increase, a 6-fold increase, a 7-fold increase, a 8-fold increase, a 9-fold increase, a 10-fold increase, a 11-fold increase, a 12-fold increase, a 13-fold increase, a 14-fold increase, a 15-fold increase, a 16-fold increase, a 17-fold increase, a 18-fold increase, a 19-fold increase, a 20-fold increase, a 25-fold increase, etc.

In some instances, relative levels may be computed by determining a ratio of the level one or more metabolites to the level of one or more compounds from which the one or more metabolites are produced.

As summarized above, the methods of the present disclosure may be employed to detect a neuroaxonal dystrophy associated with vitamin E deficiency in a non-human subject. In some embodiments, the methods find use in assessing horses. In some instances, the methods may be employed to detect a neuroaxonal dystrophy associated with vitamin E deficiency disorder in a horse. In some instances, the methods may be employed, e.g., to detect equine neuroaxonal dystrophy (eNAD), equine degenerative myeloencephalopathy (EDM) or both or related conditions. In some instances, horses of interest may include those in which eNAD and/or EDM have been reported or are at an increased risk of eNAD and/or EDM. In some instances, the methods may find use in assessing one or more particular breeds of horse or the offspring of crosses of particular breeds of horse, including but not limited to e.g., one or more of Quarter horses/Paints/Appaloosas, Haflingers, Standardbreds, Thoroughbreds, Ponies (e.g., Pony of the Americas), Lusitano/Andalusians, Morgans, Paso Finos, Arabians, Tennessee Walking Horse, Norwegian Fjord, and/or various Mixed Breeds.

Horses, or other non-human animals, evaluated according to the herein described methods will vary in age and may range from 6 months or less to 5 years or older including but not limited to e.g., under 6 months, birth to 6 months, birth to 1 year, 6 months to 1 year, birth to 2 years, 6 months to 2 years, 1 year to 2 years, over two years, 2 years to 5 years, over 5 years, etc. Horses, or other non-human animals, evaluated according to the herein described methods may or may not display symptoms of eNAD and/or EDM at the time of assessment and/or prior to the time of assessment. Put another way, in some instances, the methods may be employed to screen an animal that does not display symptoms of the disorder.

Screening may be performed at any convenient and appropriate time, including but not limited to e.g., after a known period of vitamin E deficiency, after a suspected period of vitamin E deficiency, at or within hours of birth, within 6 hours of birth, within a day of birth, within a week of birth, within a month of birth, within 2 months of birth, within 3 months of birth, within 6 months of birth, between birth and 6 months, between and 1 year, at 6 months to 1 year of age, between birth and 2 years, at 6 months to 2 years of age, at 1 year to 2 years or age, at over two years of age, at 2 years to 5 years of age, at over 5 years of age, etc. In some instances, a single round of screening may be performed. In some instances, screening may be performed at regular intervals, including e.g., regular intervals within any of the above described time periods, including but not limited to e.g., semiannually, annually, quarterly, bimonthly, monthly, biweekly, weekly, every other day, daily, and the like. In some instances, screening may be performed for one or more particular breeds of horse, including e.g., those breeds which may have increased susceptibility (e.g., based on heredity and/or environmental and/or dietary factors) to neuroaxonal dystrophies associated with vitamin E deficiency above that of other breeds and/or horses in general.

In some instances, the methods may be employed to assess an animal that does display symptoms of the disorder, but to which a diagnosis has not yet been assigned. For example, in some instances, a practitioner (e.g., a veterinarian or other animal health professional) may assess an animal that has been reported to show, or currently displays, one or more symptoms of a neuroaxonal dystrophy associated with vitamin E deficiency. The practitioner may directly obtain a sample from the subject or a sample may be provided to the practitioner (e.g., from the owner or other caregiver). Similarly, for screening purposes, e.g., where symptoms may not be present or suspected, the practitioner may directly obtain a sample from the subject or a sample may be provided to the practitioner (e.g., from the owner or other caregiver).

In some embodiments, the practitioner may take a baseline sample (or baseline measurement) of vitamin E and/or one or more vitamin E metabolites. In some instances, whether a baseline sample or measurement is acquired, the practitioner may administer a bioavailable alpha-tocopherol, including e.g., where the bioavailable alpha-tocopherol is administered by injection. At some period of time, including e.g., 30 min., 1 hr, 2, hr, 3 hr, 4 hr, 5 hr, 6 hr, 12 hr, 18 hr, 24 hr, 30 hr, 36 hr, 42 hr, 48 hr, 3 d, 4 d, 5 d, 6 d, 7 d, 1.5 weeks, 2 weeks, etc., following the administration of the bioavailable alpha-tocopherol, one or more testing samples or one or more testing measurement may be taken to analyze the level of one or more alpha-tocopherol metabolites in the sample. In some instances, an obtained sample (including e.g., test samples and baseline samples) may be shipped for testing. In some instances, samples, including shipped samples, may be protected from light, kept cold (e.g., on ice or dry ice), or a combination thereof.

In some instances, employed methods may include one more additional tests, i.e., in addition to the methods of assessment as described herein. Non-limiting examples of additional tests may include gait analysis, cervical vertebral malformation (wobblers) testing, equine protozoal myeloencephalitis (EPM) testing, or spinal trauma evaluation, and the like. Other useful examples of additional testing may include but are not limited to e.g., neurologic examination, radiographs of the vertebrae of the neck, spinal tap, and the like. Animals assessed according to the methods described herein, with or without additional testing as described herein, may or may not have gait abnormalities. For example, in some instances, an assessment of vitamin E metabolism may be performed on a subject that tests normal for one or more of the additional tests described herein. In some instances, an assessment of vitamin E metabolism may be performed on a subject having a test result that suggests an abnormality for one or more of the additional tests described herein, including e.g., where the abnormality suggests or is consistent with a neuroaxonal dystrophy associated with vitamin E deficiency.

As summarized above, the methods of the present disclosure may include measuring a metabolite level in a sample from a non-human subject, such as a horse, including e.g., a horse of a specific breed. Any useful and convenient sample may be employed including but not limited to e.g., a biological sample.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides or polypeptides. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” may also include, in some instances, solid tissue samples, tissue culture samples, and cellular samples.

In some instances, an assayed sample may be serum, plasma or urine. Such samples may be collected by any convenient method and according to various time schedules. In some instances, samples used in analysis may be collected within 48 hours or less from administration of a bioavailable alpha-tocopherol, including but not limited to e.g., 36 hours or less, 24 hours or less, 12 hours or less, 6 hours or less, from 6 to 24 hours, from 12 to 36 hours, from 12 to 24 hours, etc.

A biological sample is obtained from any subject (e.g., equine subject) to be tested as described herein, including, e.g., an equine breed described herein or a mixed breed. In some embodiments, the equine is a neonate, a colt or a foal. A biological sample can be suspended or dissolved in liquid materials such as buffers, extractants, solvents and the like. A biological sample can be separated or isolated from its component parts, e.g., serum or plasma may be separated from a whole blood sample. In some embodiments, a blood sample containing metabolites of interest is centrifuged to separate serum or plasma from other blood components.

The biological sample may be obtained from an equine exhibiting one or more symptoms of eNAD and/or EDM. In some embodiments, the equine is asymptomatic, but is suspected of being predisposed to developing eNAD and/or EDM, e.g., due to breed, parentage or lineage. In some embodiments, the biological sample is from an equine who has a parent, grandparent or sibling that is or has suffered from eNAD and/or EDM. In some embodiments, a sample may be obtained from an equine that is suspected of being vitamin E deficient. Animals suspected of being vitamin E deficient may or may not display one or more symptoms of vitamin E deficiency and/or may be suspected of having a diet that is vitamin E deficient and insufficient to provide the animal with the necessary levels of dietary vitamin E. In certain embodiments, a biological sample is also obtained from an equine that is not suffering from or suspected of developing eNAD and/or EDM as a negative control. In certain embodiments, a biological sample is also obtained from an equine known to be suffering from eNAD and/or EDM as a positive control.

As summarized above, the methods of the present disclosure may include administering to the subject a bioavailable alpha-tocopherol. Administration of bioavailable alpha-tocopherol may be performed for a variety of reasons, including e.g., to assess tocopherol metabolism in the subject, to treat the subject for a detected vitamin E deficiency and/or a neuroaxonal dystrophy associated with vitamin E deficiency, and the like.

Any convenient route of administration may be employed including but not limited to e.g., oral administration, injection, and the like. In some embodiments, e.g., where the metabolism of an alpha-tocopherol is assessed, direct injection administration may be employed, including e.g., a single injected administration. In some embodiments, e.g., where the metabolism of an alpha-tocopherol is assessed, oral administration may be employed, including e.g., a single oral administration. In some instances, multiple administrations (e.g., 2, 3, 4, 5, 6, etc.) may be employed, including but not limited to e.g., where the subject is administered a tocopherol in order to treat the subject for a vitamin E deficiency or related disorder, as described in more detail below. Depending on the context, any convenient bioavailable alpha-tocopherol may be employed. Generally, particularly for administration employed to evaluate tocopherol metabolism, the administered bioavailable alpha-tocopherol is a form that is sufficiently metabolized for convenient metabolite detection. In some instances, RRR-alpha-tocopherol may be employed. The concentration at which the bioavailable alpha-tocopherol is administered may also vary, e.g., depending on the context and/or the objective of the administration. Non-limiting useful concentrations of administration may include but are not limited to e.g., from 2500 to 10,000 IU/450 kg, from 4000 to 6000 IU/450 kg, and the like.

In the following, variations on metabolite assessment, including e.g., pre- and post-administration assessments are described with specific reference to measurement of alpha-CMBHC. However, such variations are not intended to be limited to measurement of alpha-CMBHC and may, in some instances, be understood to also apply to assessments of one or more other metabolites and/or one or more tocopherols and/or one or more tocotrienols, e.g., in place of or in combination with the described alpha-CMBHC measurement, as appropriate.

In some instances, the metabolite assessment of a subject method may be solely a post-administration measurement of alpha-CMBHC. In some instances, a subject method may include additional metabolite assessments, including addition pre- and/or post-administration assessments. In some instances, a subject method may include measuring a pre-administration alpha-CMBHC concentration in a sample from the subject. In some instances, a subject method may include comparing the post-administration alpha-CMBHC concentration to the pre-administration alpha-CMBHC concentration. In some instances, a subject method may include measuring a pre-administration alpha-tocopherol concentration in a sample from the subject. In some instances, a subject method may include measuring a post-administration alpha-tocopherol concentration in a sample from the subject. In some instances, a subject method may include comparing the post-administration alpha-tocopherol concentration to the pre-administration alpha-tocopherol concentration.

In some instances, the subject methods may include measuring a plurality of post-administration alpha-CMBHC concentrations. Such a plurality of measurements may e.g., include where each measurement is performed at a different timepoint following administration of the alpha-tocopherol. In some instances, a subject method may include measuring at least a 6 hour post-administration timepoint and a 12 hour post-administration timepoint. In some instances, a subject method may include measuring a plurality of post-administration alpha-tocopherol concentrations, or metabolites thereof, each at a different timepoint following the administration. In some instances, a subject method may include measuring at least a 6 hour post-administration timepoint and a 12 hour post-administration timepoint.

In some instances, the sample may be prepared prior to measuring a level of one or more vitamin E metabolites (e.g., one or more alpha-tocopherol metabolites) and/or one or more vitamin E isoforms. Sample preparation may in some instances include extraction of one or more vitamin E metabolites (e.g., one or more alpha-tocopherol metabolites) and/or one or more vitamin E isoforms from the sample. In some instances, frozen samples, such as e.g., frozen plasma and serum samples, may be placed in a refrigerator at 4° C. until thawed as part of sample preparation. Sample preparation may further include, in some instances, mixing (e.g., inversion, shaking, homogenization, etc.), aliquoting, filtering, and the like.

Sample extraction may, in some instances, include phospholipid removal, including but not limited to e.g., where proteins and/or phospholipids are removed from plasma samples without affecting target analyte recovery. Extraction may further involve one or more solvents, buffers, antioxidants such as but not limited to e.g., acetonitrile, water, ethanol, isopropanol, methanol, ascorbic acid, butylated hydroxytoluene, combinations thereof and the like. In some instances, samples may be extracted under vacuum and/or may be dried (e.g., lyophilized) following extraction. In some instances, e.g., where an extracted sample/analyte is dried, the sample/analyte may be reconstituted in an appropriate reconstitution solution, including but not limited to e.g., where the sample/analyte is reconstituted in methanol. Preparation of the reconstituted sample may, in some instances, include vortexing, sonication, centrifugation, and combinations thereof.

In some instances, one or more internal standards may be included in a test sample, including but not limited to e.g., where the one or more internal standards are “spiked into” the test sample. An internal standard may be of known amount or concentration and provide a control and/or reference against which the tested analyte may be evaluated. Useful internal standards include but are not limited to e.g., α-tocopherol (α-TP)-D6 standard, chlorpropamide standard, and the like, including where such standards are dissolved in an appropriate solvent, such as e.g., methanol. Internal standards may be added at various time points in a procedure, including e.g., where such standards are added prior to extraction.

Any convenient method of measuring the level and/or concentration of the tocopherols and/or metabolites thereof assessed in the subject methods may be employed. Useful methods of measuring vitamin E and vitamin E metabolites include but are not limited to e.g., methods employing UV-VIS and various forms of mass spectrometry, including e.g., GC/MS and LC/MS/MS analysis. In some instances, a subject method may employ liquid chromatography-mass spectrometry (LC-MS) to measure the level(s) and/or concentration(s) of the one or more tocopherols and/or metabolites thereof assessed in the methods. In some instances, a panel of tocopherols and/or metabolites thereof may be assessed in a subject method. For example, a panel including any combination of alpha-CMBHC, alpha-tocopherol (α-TOH), gamma-tocopherol (γ-TOH), alpha-tocotrienol (α-TOT), gamma-tocotrienol (γ-TOT), alpha-carboxyethylhydroxychroman (α-CEHC), gamma-carboxyethylhydroxychroman (γ-CEHC), and the like, may be employed. Measuring the various member of a panel may vary and may include but is not limited to e.g., simultaneous measurement of the members of the panel, sequential measurement of the members of the panel, and the like. Where a panel of analytes is employed, the assessment employed may or may not include all of the analytes of the panel in the assessment. For example, in some instances, only one member or a subset of the members of the panel may be employed in the method of assessing or detecting a disorder.

In some instances, analysis of vitamin E and vitamin E metabolites may employ an ultra-performance liquid chromatography-electrospray mass spectrometry (UPLC-ESI/MS/MS) system. Any convenient UPLC-ESI/MS/MS system may be employed including but not limited to e.g., those commercially available from spectrometer Bruker Corp, (Fremont, Calif., USA), such as but not limited to e.g., Bruker Advance UPLC system coupled with Bruker EVOQ Elite MS/MS triple quadrupole mass spectrometer. In some instances, analytes may be monitored by multiple reaction monitoring (MRM), including e.g., where tocopherols and tocotrienols are monitored in positive mode and metabolites are monitored in negative mode. In some instances, the most abundant product ion of an analyte may be selected for quantification. In some instances, two or more analytes may be selected for quantification, including but not limited to e.g., where two or more abundant ions are selected for confirmation of a most abundant ion quantification. Useful buffers for LC-MS conditions include but are not limited to e.g., MeOH/water 50:50 with 0.1% formic acid, MeOH/water 50:50 with 0.1% acetic acid, MeOH/water 50:50 with 1.0% acetic acid, and the like.

In some instances, analysis may include the establishment of a standard curve. For example, in some instances, calibration standards may be prepared in a suitable buffer (e.g., methanol) at a range of concentrations (e.g., 0.1, 0.5, 1, 5, 10, 50, 100, 500, 1,000, 2,000, 5,000, and 10,000 ng/mL) for all (or a portion of, or one) of the analytes to be tested. In some instances, internal standards may also be used in preparation of a standard curve. Following preparation, the calibration standards may be run on the instrument under analysis parameters and the values produced may be used to establish a standard curve for one or more (including all) of the analytes.

In some embodiments, following sample analysis, a measured level of one or more vitamin E metabolites, with or without measurement of one or more vitamin E isoforms, may be employed to inform one or more further decisions or actions towards the subject from which the sample was derived. Such decision and/or actions include but are not limited to e.g., one or more treatment decision, one or more breeding decisions, and the like.

As summarized above, the present disclosure also includes methods of treating a non-human subject for a neuroaxonal dystrophy associated with vitamin E deficiency. Such methods may include detecting the presence or absence of the neuroaxonal dystrophy associated with vitamin E deficiency in the non-human subject including e.g., where such detection may include any of those methods, and aspects thereof, described above. In some instances, following detection of the presence of a neuroaxonal dystrophy associated with vitamin E deficiency, the subject may be treated with vitamin E supplementation, including but not limited to e.g., oral, dietary, injected, or intravenous vitamin E supplementation. Such treatments may apply to a variety of subjects. For example, in some instances with regards to equine subjects, a foal may be treated with vitamin E supplementation to treat the symptoms of, or prevent the onset of, a neuroaxonal dystrophy associated with vitamin E deficiency detected based on the outcome of an assessment described herein. With further regard to equine subjects, adult horses who have been vitamin E deficient for a period of 18 months can develop lower motor neuron weaknesses. Thus, in some instances, an adult horse that has been deprived, or is suspected of having been deprived, of sufficient vitamin E may be treated with vitamin E supplementation to treat the symptoms of, or prevent the onset of, a neuroaxonal dystrophy associated with vitamin E deficiency detected based on the outcome of an assessment described herein.

As summarized above, the present disclosure also includes methods of treating a subject for an eNAD/EDM disorder. Such methods will generally include detecting whether the subject has an eNAD/EDM disorder, including e.g., where such detection may include any of those methods, and aspects thereof, described above. Once the subject is determined to have an eNAD/EDM disorder, e.g., through employment of a detection method as described herein, the subject may then be treated for the disorder. For example, in some instances, a subject having an eNAD/EDM disorder may be administered high dose alpha-tocopherol to treat the eNAD/EDM disorder. As a non-limiting example, in some instances, high dose vitamin E may be at least 5 IU/kg/day, including but not limited to e.g., 6 IU/kg/day, 8 IU/kg/day, 10 IU/kg/day, etc. In some instances, the subject may be young, including e.g., in the case of horses the subject may be 2 years of age or less, 1.5 years of less, 1 year or less, 6 months or less, etc.

In some instances, administration of tocopherol may be continued for a pre-determined period of time, e.g., according to a pre-determined treatment schedule, over one or more days, weeks, months, years, etc., including but not limited to e.g., 2 or more days, 3 or more days, 4 or more days, 5 or more days, a week or more, two weeks or more, 3 weeks or more, a month or more, two months of more, three months or more, four months or more, six months or more, eight months or more, ten months or more, a year or more, two years or more, three years or more. In some instances, a subject may be administered an amount of vitamin E over the time period at a particular frequency, including but not limited to e.g., where the administration frequency is twice daily, daily, every other day, every third day, weekly, bi-weekly, monthly, etc. In some instances, the amount administered over the period of time at the desired frequency may range from 2500 to 10,000 IU/450 kg, from 4000 to 6000 IU/450 kg of vitamin E or be 2,500 units, 3,000 units, 3,500 units, 4,000 units, 4,500 units, 5,000 units, etc., of vitamin E.

As summarized above, the present disclosure also includes methods of screening a non-human subject for breeding purposes. For example, in some instances, a non-human subject may be screened to detect whether the subject has an eNAD/EDM disorder prior to being employing in breeding processes to produce offspring. Such practices may prevent passing an eNAD/EDM disorder on to any generated offspring. Accordingly, when a subject is assayed and an absence of an eNAD/EDM disorder is detected, then the subject may be employed in further breeding procedures. In some instances, a subject animal may be tested prior to maturity (i.e., before breeding age) in order to inform a later breeding decision and/or action performed or made when the animal has reached breeding age.

Various breeding procedures may be employed. For example, in some instances, the breeding procedures may include artificial insemination, including in some instances, semen collection and/or storage, ova collection and/or storage, and the like. In embodiments where the subject is a horse, breeding procedures may include live cover or one or more advanced reproductive techniques. Various advanced reproductive techniques may be employed, alone or in combination, including but not limited to e.g., embryo transfer, gamete intrafallopian transfer (GIFT), egg transfer and intracytoplasmic sperm injection (ICSI).

### Kits, Reagents and Devices

Aspects of the present disclosure also include kits, reagents and/or devices for use in practicing the herein described methods. The kits may include, e.g., one or more components and/or reagents and/or devices, where applicable, for practicing one or more of the above-described methods. The subject kits may vary greatly. Kits of interest include those having one or more reagents mentioned herein, and associated devices where applicable, with respect to the methods of detecting and/or treating and/or breeding based on the detection of an eNAD/EDM disorder, and the like.

In some instances, useful kits may include one or more doses (whether or not in unit dosage form) of bioavailable alpha-tocopherol for administration to a non-human subject. For example, in instances where the dose(s) is orally administered, a subject kit may include one or more doses of bioavailable alpha-tocopherol configured for oral administration and in an amount compatible with the above described methods. In instances where the dose(s) is injection administered, a subject kit may include one or more doses of bioavailable alpha-tocopherol configured for injection and in an amount compatible with the above described methods. In some instances, a subject kit may include one or more specimen or sample collection containers, e.g., for collecting one or more pre- or post-alpha-tocopherol-administration samples from the non-human subject. Various sample collection containers may be employed, e.g., depending on the type of sample collected. In some instances, a subject collection container may be configured for shipping the specimen to a facility for analysis, e.g., an eNAD/EDM disorder detection method as described herein.

In addition to the above components, the subject kits may further include (in some embodiments) instructions for practicing the subject methods (or instructions for practicing one or more portions of the subject methods such as e.g., agent administration, sample collection, etc.). These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., diskette, compact disk (CD), portable flash drive, Hard Drive etc., on which the information has been recorded. Yet another form of these instructions that may be present is a website address which may be used via the internet to access the information at a removed site.

The methods, kits, reagents, device and the like, as described above, may provide for certain advantages, which are not intended to be limiting. For example, the instant methods provide for the antemortem detection of neuroaxonal dystrophy associated with vitamin E deficiency in non-human subjects. Such antemortem methods have clear advantages over postmortem methods of detection, including providing the opportunity to make certain decisions and/or perform certain actions with respect to the non-human subject, including treatment decisions and/or actions, breading decisions and/or actions and the like. Moreover, early detection, including e.g., pre-symptomatic detection, of neuroaxonal dystrophy associated with vitamin E deficiency in non-human subjects may provide further advantages with regards to treatment, including e.g., the ability to begin treatment at an early stage which may provide improved in treatment outcomes as compared to treatment begun at a later stage or timepoint. Such advantages are merely exemplary, non-exhaustive, and not intended to be limiting.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene (Agilent Technologies), Invitrogen (Thermo Fisher Scientific), Sigma-Aldrich, and Clontech (Takara Bio USA, Inc.).

### Example 1: Vitamin E Metabolism in Horses with Equine Neuroaxonal Dystrophy

Vitamin E (vitE) functions as a biological antioxidant, preventing the oxidation of unsaturated lipids within cellular and subcellular membranes by neutralizing production of free radicals. Through this mechanism, and potentially other mechanisms yet to be elucidated, vitE serves to maintain normal neuromuscular function. Several specific equine diseases develop in the face of vitE deficiency. These include nutritional myodegeneration, equine neuroaxonal dystrophy and equine degenerative myeloencephalopathy (eNAD/EDM) in young animals. Adult horses deficient in vitE may develop a vitE deficient myopathy or equine motor neuron disease. Treatment with vitE is usually instituted in an attempt to reverse clinical signs. However, selecting the type and amount of vitE to supplement can be challenging for veterinarians because the bioavailability and potency varies widely among commercial supplements. In addition, for progressively degenerative disorders, such as eNAD/EDM, it may be desirable to begin supplementation before disease symptoms are evident, thus preventing disease progression. However, pre-symptomatic supplementation is often not possible, particularly without, or an unknown, genetic predisposition to eNAD/EDM, as current methods of diagnosis are largely based on gross observation of anatomical and behavioral traits or postmortem analysis.

Most vitE supplements consist of natural or synthetic forms of alpha-tocopherol (α-TOH) because it is the most biologically and well researched isoform of vitE. Vitamin E, however is a complex nutrient consisting of eight closely-related fat-soluble naturally occurring compounds that form two groups; tocopherols (saturated) and tocotrienols (unsaturated). Within each group, there are four individual isoforms (α, β, γ and δ).

Studies involving the metabolism of alpha-tocopherol in normal horses and horses having equine neuroaxonal dystrophy (eNAD) or equine degenerative myeloencephalopathy (EDM) were undertaken. Equine Vitamin E deficient diseases generally fall into either Early-onset (6-24 months) or Late-onset categories. Late-onset Equine Vitamin E deficient diseases include Vitamin E-deficient myopathy (7-10 years) and Equine Motor Neuron Disease (greater than 10 years, peaking at 16 years). eNAD/EDM are Early-onset Equine Vitamin E deficient diseases that include spinal ataxia and have an underlying genetic basis. Definitive diagnosis of eNAD/EDM conventionally requires histologic evaluation of the CNS (postmortem). Supportive eNAD diagnostic criteria include: young (typically less than 3 years), a duration of static or progressive clinical signs for greater than 2 weeks, unremarkable cervical films/myelogram, and negative equine protozoal myeloencephalitis (EPM) test. Important factors to consider in diagnosis may also include a history of ataxia in siblings and/or half-siblings and low serum alpha-tocopherol; however, in many instances, serum alpha-tocopherol levels may be normal.

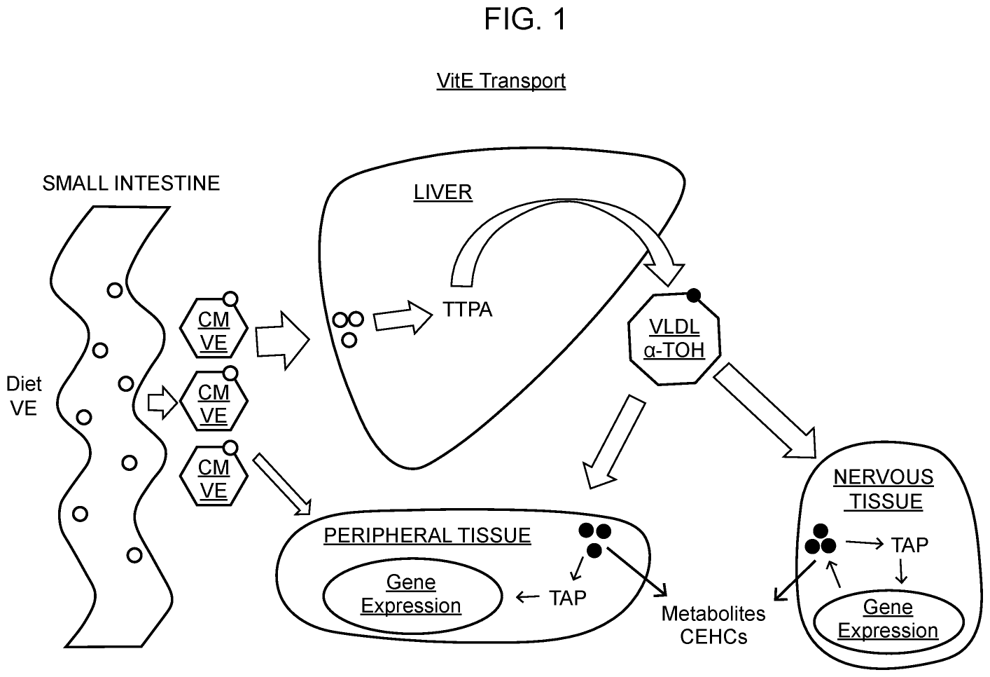

Differences in plasma α-TOH levels observed in eNAD-affected and unaffected horses have been suspected to be caused by genetic mutations in genes involved in the absorption, transport or catabolism of vitE. For reference, a schematic depiction of Vitamin E transport is provided in FIG. 1. Across species, vitE is absorbed through the small intestine after solubilization by bile acids and is then transported in the circulation in chylomicrons via the lymphatic system. In an earlier study, α-TOH absorption was measured in eNAD-affected and unaffected horses and no significant differences were observed in the absorption indices. Transport of α-TOH is lipoprotein-associated and it has been previously determined that variation in plasma α-TOH in eNAD-affected horses does not correlate with variation in plasma lipoprotein concentrations.

VitE is delivered to the liver, where the α-TOH isoform is integrated into very low-density lipoproteins by TTP before being transported back into circulation. It is important to note that TTP has a higher binding affinity for α-TOH and thus, its binding affinity for other forms of vitE is determined by the levels of α-TOH present. Therefore, high levels of α-TOH lead to an increase in the catabolism of non-α-TOH forms of vitE. Once in circulation, lipoproteins deliver α-TOH to different locations in the body. In regard to the hepatic uptake and transport of vitE, the lower α-TOH concentrations observed in eNAD-affected horses are not caused by a genetic mutation in TTPA, as is the case in humans.

As eNAD is a disease affecting the central nervous system, the brain and spinal cord are important anatomic regions to study the effect of vitE on eNAD-affected horses. Therefore, to complete the transport process of α-TOH to the brain and spinal cord, where it then can perform required antioxidant functions, it must first cross the blood-brain barrier (BBB). Transport across the BBB in eNAD-affected horses appears to be comparable to healthy horses.

While α-TOH absorption and transport in eNAD-affected horses do not appear to be altered, α-TOH plasma concentrations are frequently lower. However, these differences alone have not proved diagnostically useful. Without being bound by theory, this study was undertaken to investigate whether a difference exists in the way in which α-TOH is catabolized in these horses.

This example describes investigations into the role of metabolism of α-TOH in horses affected with eNAD/EDM. Accordingly, the rate of α-TOH and γ-TOH metabolism was determined through the measurement of CEHCs in healthy and eNAD-affected horses following α-TOH supplementation.

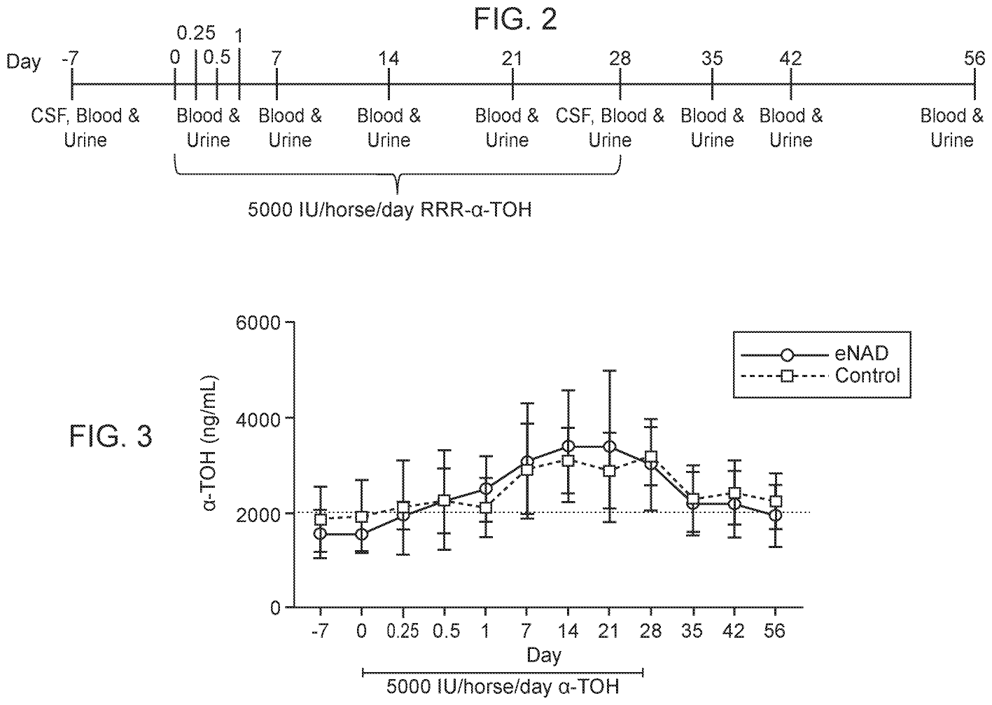

In particular, the levels of a panel of tocopherols and tocopherol metabolites were assessed by liquid chromatography-mass spectrometry (LC-MS) in samples obtained from eNAD affected horses and matched healthy controls. Reference ranges of α-TOH, γ-TOH, α-TOT, γ-TOT, α-CEHC, γ-CEHC, and α-CMBHC were determined from serum, plasma and urine samples obtained from 16 healthy adult Thoroughbred horses on pasture. Next a supplementation trial was performed employing samples from eNAD-affected horses and neurologically normal age and sex-matched controls. Levels were assessed at baseline and after administration of bioavailable RRR-alpha-tocopherol according to the sample collection schedule described (see e.g., FIG. 2).

Analytes were simultaneously quantified via ultra-performance liquid chromatography-tandem mass spectrometry (LC-MS). LC-MS analysis was carried out using a Bruker EVOQ LC-TQ Mass Spectrometer coupled with a Bruker Advance HPLC system (Bruker Corp, Freemont, Calif., USA). Mass spectral data for α- and γ-tocopherols and α- and γ-tocotrienols were acquired in the positive ion electrospray ionization (ESI) mode with the following retention times (RT) and scan events: γ-tocotrienol—RT 11.65 min, MRM 411.0>151.0 (CE 19.0V), α-tocotrienol—RT 12.0 min, MRM 425.1>165.1 (CE 20.0V), γ-tocopherol—RT 13.3 min, MRM 417.2>151.0 (CE 25.0V), 5-methyl-D3,7-methyl-D3 (TD6)—RT 13.8 min, MRM 437.4>171.0 (CE 27.0V), and α-tocopherol—RT 13.8 min, MRM 431.2>165.0 (CE 25.0V). Mass spectral data for α- and γ-CEHC and α-CMBHC were acquired in the negative ion ESI mode with the following retention times (RT) and scan events: α-CMBHC—RT 7.45 min, MRM 319.1>150.1 (CE 22.0V), α-CEHC—RT 5.70 min, MRM 277.0>233.1 (CE 14.0V), and γ-CEHC—RT 5.30 min, MRM 263.1>219.1 (CE13.0V). Quantification was carried out using a 10-point calibration curve covering the range from 0.1 ng/mL to 5,000 ng/mL and linear regression. Each level of calibration standard contained the internal standard TD6 at 100 ng/mL and CUDA at 2 ng/mL, matching their final concentrations in the analysis samples. Analysis of negative control reagent blanks for tocopherols by the LC-MRM technique described above showed clean chromatograms with no background contribution from contaminations.

The limit of detection (ng/mL) and detected range (ng/mL), respectively, for each plasma vitamin E metabolite measured in these initial trials were as follows: α-TOH (0.1; 500-1000), γ-TOH (0.2; 10-15), α-tocotrienol (0.2; 0.2-0.6), γ-tocotrienol (0.2; 5-10), α-CMBHC (<0.05; 0.1-0.4), γ-CEHC (<0.05; 0.2-0.6), and α-CEHC (<0.05; 0.1-0.2).

Simultaneous quantification of α-TOH, γ-TOH, α-tocotrienol, γ-tocotrienol, α-CEHC, γ-CEHC, and α-CMBHC has not been previously performed in the horse. Data was Log-transformed. Correspondence between sample types (e.g., plasma vs. serum) was evaluated and statistical testing by Mixed-model ANOVA was performed (fixed variables=disease, time; randomized variable=horse; 2-way interactions; p<0.05).

With regards to the correspondence between sample types, the results demonstrated that all values between plasma and serum were highly correlated (P<0.0001); however, α-TOH values were slightly higher. Despite these the high degree of correlation between sample types, the sample types were used for comparisons across time points in the further analysis.

In addition, whether administration of medications can affect analyte levels was also evaluated using measured analyte values before (Pre-) and after (Post-) anesthetic administration at day 28 for both plasma and serum samples. The results of Pre- vs. Post-anesthesia analyte level difference testing are provided in the following Table 3:

TABLE 3

| Analyte | Plasma | Serum |
|---|---|---|
| α-TOH | NS | NS |
| γ-TOH | NS | NS |
| α-TOT | NS | NS |
| γ-TOT | P = 0.03 (most values below RL) | NS |
| α-CEHC | P = 0.02 (higher post-anesthesia) | NS |
| γ-CEHC | P = 0.0005 (higher post-anesthesia) | NS |
| α-CMBHC | NS | NS |

From the results presented above it was determined that administration of medications can, in some instances, affect analyte levels and that, if any other medications are being co-administered, serum samples would be used.

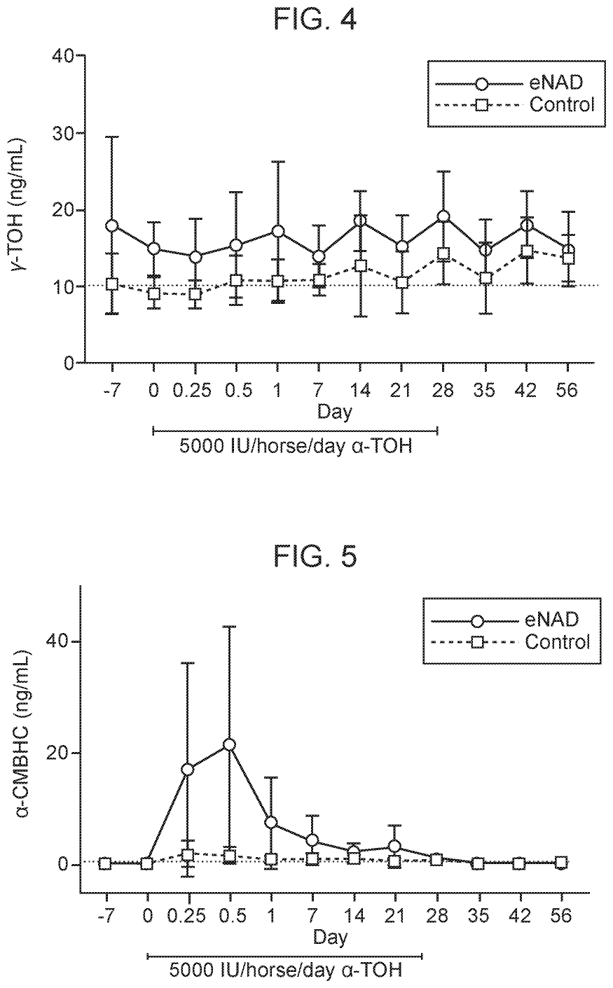

Serum α-TOH and γ-TOH levels are shown in FIG. 3 and FIG. 4, respectively, with box plots for NAD and control (left and right, respectively, for each pair of box plots) representing the measured values from the corresponding samples. For reference, the mean, median and range of α-TOH values for healthy grazing Thoroughbreds (TBs) (N=16) are: 4292±892, 4348, and 2855-6299, respectively. The mean, median and range of γ-TOH values for healthy grazing TBs (N=16) are: 27.1±9.82, 25.6, and 10.3-44.7, respectively.

A summary of serum [α-TOH] values during the trial are summarized below in Table 4:

TABLE 4

| Serum α-TOH | Control Horses | eNAD Horses |
|---|---|---|
| (μg/mL) | (median, range) | (median, range) |
| Baseline | 1.76 (0.94-2.85) | 1.77 (0.79-1.97) |
| Peak serum Day 14 | 3.32 (1.93-3.87) | 2.93 (2.34-4.72) |
| Depletion Day 56 | 1.96 (1.78-3.21) | 1.77 (1.39-2.99) |

Low amounts of tocotrienols were detected in serum samples and no significant difference in α-TOT or γ-TOT levels were seen with time or disease. Mean, median, and range values for α-TOT (ng/ML) in healthy grazing TBs were: 0.31±0.36, 0.15, and 0.004-1, respectively. Mean, median, and range values for γ-TOT (ng/ML) in healthy grazing TBs were: 0.13±0.36, 0.02, and 0.02-1.79, respectively.

Serum α-CMBHC levels are shown in FIG. 5 with box plots for NAD and control (left and right, respectively, for each pair of box plots) representing the measured values from the corresponding samples. For reference, the mean, median and range of α-CMBHC values for healthy grazing TBs (N=16) are: 0.72±0.37, 0.6, and 0.32-1.71, respectively. Serum α-CMBHC values during the trial, as a measure of α-TOH metabolism, are summarized in Table 5 below and serum α-CMBHC fold increases in eNAD as compared to control horses are summarized in Table 6 below (***P<0.001, **P<0.01, *P<0.05):

TABLE 5

| | | Control Horses | eNAD Horses |
|---|---|---|---|
| Analyte | Time Point | (median, range) | (median, range) |
| Serum | Baseline | 0.22 (0.17-0.36) | 0.24 (0.17-0.47) |
| α-CMBHC | Peak-Day 0.5 | 1.11 (0.64-4.61)* | 13.0 (0.73-53.9)* |
| (ng/mL) | Depletion-Day 56 | 0.28 (0.15-0.35) | 0.32 (0.12-0.40) |

TABLE 6

| Time post α-TOH | eNAD | Control Horses |
|---|---|---|
| administration | (median, range) | (median, range) |
| 6 hr | 20.7** | 4.26 |
| | (4.41-162.4) | (0.96-34.45) |
| 12 hr | 53.3*** | 5.45 |
| | (4.28-152) | (1.65-24.29) |
| AUC (0.24 h) | 14.1 ± 7.07* | 1.34 ± 0.59 |

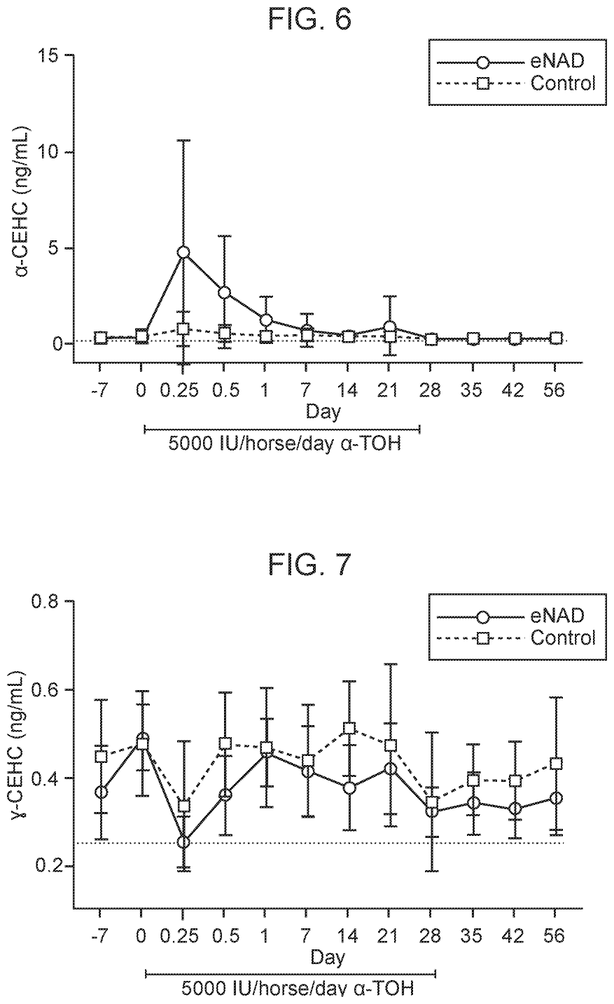

Serum α-CEHC levels are shown in FIG. 6 with box plots for NAD and control (left and right, respectively, for each pair of box plots) representing the measured values from the corresponding samples. For reference, the mean, median and range of α-CEHC values for healthy grazing TBs (N=16) are: 0.33±0.12, 0.31, and 0.18-0.67, respectively.

Serum γ-CEHC levels are shown in FIG. 7 with box plots for NAD and control (left and right, respectively, for each pair of box plots) representing the measured values from the corresponding samples. For reference, the mean, median and range of γ-CEHC values for healthy grazing TBs (N=16) are: 0.43±0.18, 0.38, and 0.26-0.89, respectively.

These data, and particularly α-CMBHC and α-CEHC levels at 6 hours and 12 hours post-supplementation, demonstrate that eNAD horses metabolize α-TOH at faster rates than control horses. Correspondingly, these examples show that measuring Vitamin E metabolites following Vitamin E supplementation is a useful assay for highly sensitive antemortem detection eNAD specifically and Vitamin E-associated neuromuscular diseases generally.

From these findings a protocol and assay to assess the rate of metabolism of alpha-tocopherol (vitamin E) in horses and the presence of eNAD was devised. Based on performed studies, this test is able to diagnose equine neuroaxonal dystrophy (eNAD), a devastating inherited neurologic disease in horses that, to date, has only been diagnose-able via necropsy after euthanasia. An antemortem diagnostic test for eNAD provides owners and veterinarians with the ability to diagnose these horses while still living. The results from such tests allow owners and veterinarians to make informed decisions regarding breeding or euthanasia. While there is no curative treatment for eNAD once a horse is over 2 years of age, horses 2 years of age or younger can be treated to reduce symptoms and slow the rate of disease onset. Achieving an antemortem definitive diagnosis for this disease greatly benefits the equine industry.

An exemplary protocol for assessing the rate of metabolism of alpha-tocopherol involves the following: (1) Measure baseline serum alpha-tocopherol and alpha-CMBHC concentrations (i.e. time point=0); (2) Administer 5000 IU/450 kg horse of RRR-alpha-tocopherol orally once; (3) Reassess serum alpha-tocopherol and alpha-CMBHC concentrations at 6 (i.e. time-point=6 h) and 12 hrs (i.e. time-point=12 h). From these results, if the concentration of alpha-CMBHC is >9 ng/mL at either time point, an eNAD is likely present.

The following Table 7 provides the fold-increases in alpha-CBMHC seen in eNAD subjects and healthy controls at 6 hour and 12 hour timepoints:

TABLE 7

| | | Mean | SD | Min | Max |
|---|---|---|---|---|---|
| eNAD/EDM | 6 h increase | 76.80168189 | 65.06375832 | 4.410404624 | 162.4771574 |
| Control | 6 h increase | 10.28491299 | 11.78741915 | 0.961325967 | 34.45405405 |
| eNAD/EDM | 12 h increase | 90.31758297 | 63.84140826 | 4.248554913 | 152.2588832 |
| Control | 12 h increase | 8.114882766 | 7.745032532 | 1.76519337 | 24.94594595 |

The following Table 8 provides equine reference ranges of alpha-tocopherol:

TABLE 8

| Serum concentration (μg/mL) | Alpha-Tocopherol Status |
|---|---|
| ≥2 | Adequate |
| 1.5-2 | Marginal |
| <1.5 | Deficient |

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered as below are provided. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of detecting a presence or absence of a neuroaxonal dystrophy associated with vitamin E deficiency in a non-human subject, the method comprising:

administering to the subject a bioavailable alpha-tocopherol;

measuring a post-administration alpha-carboxymethylbutyl hydroxychroman (alpha-CMBHC) concentration in a sample from the subject; and

detecting:
i) the presence of neuroaxonal dystrophy associated with vitamin E deficiency in the subject when the post-administration alpha-CMBHC concentration is above a predetermined threshold; or
ii) the absence of neuroaxonal dystrophy associated with vitamin E deficiency in the subject when the post-administration alpha-CMBHC concentration is below a predetermined threshold.
   2. The method according to Aspect 1, wherein the predetermined threshold is at least 9 ng/m L.
   3. The method according to Aspect 1 or 2, wherein the non-human subject is a horse.
   4. The method according to Aspect 3, wherein the neuroaxonal dystrophy associated with vitamin E deficiency is equine neuroaxonal dystrophy (eNAD)/equine degenerative myeloencephalopathy (EDM) (eNAD/EDM).
   5. The method according to Aspects 3 or 4, wherein the horse is a quarter horse, a paint/appaloosa, a haflinger, a standardbred, a thoroughbred, a pony, a lusitano/andalusian, a morgan, a paso fino, an arabian, a tennessee walking horse, a norwegian fjord, or a mixed breed.
   6. The method according to any of the preceding aspects, wherein the sample is collected within 24 hours of the administering.
   7. The method according to any of the preceding aspects, wherein the sample comprises serum.
   8. The method according to any of the preceding aspects, wherein the sample comprises plasma.
   9. The method according to any of Aspects 1 to 6, wherein the sample comprises urine.
   10. The method according to any of the preceding aspects, further comprising measuring a pre-administration alpha-CMBHC concentration in a sample from the subject.
   11. The method according to Aspect 10, wherein the method further comprises comparing the post-administration alpha-CMBHC concentration to the pre-administration alpha-CMBHC concentration.
   12. The method according to any of the preceding aspects, further comprising measuring a pre-administration alpha-tocopherol concentration in a sample from the subject.
   13. The method according to Aspect 12, further comprising measuring a post-administration alpha-tocopherol concentration in a sample from the subject.
   14. The method according to Aspect 13, wherein the method further comprises comparing the post-administration alpha-tocopherol concentration to the pre-administration alpha-tocopherol concentration.
   15. The method according to any of the preceding aspects, wherein the method comprises measuring a plurality of post-administration alpha-CMBHC concentrations each at a different timepoint following the administration.
   16. The method according to Aspect 15, wherein the method comprises measuring at least a 6 hour post-administration timepoint and a 12 hour post-administration timepoint.
   17. The method according to any of the preceding aspects, wherein the method comprises measuring a plurality of post-administration alpha-tocopherol concentrations each at a different timepoint following the administration.
   18. The method according to Aspect 17, wherein the method comprises measuring at least a 6 hour post-administration timepoint and a 12 hour post-administration timepoint.
   19. The method according to any of the preceding aspects, wherein the measuring comprises liquid chromatography-mass spectrometry.
   20. The method according to any of the preceding aspects, wherein the method comprises assessing a panel of alpha-tocopherol and metabolite levels.
   21. The method according to Aspect 20, wherein the panel of alpha-tocopherol and metabolite levels comprises alpha-CMBHC and one or more of alpha-tocopherol (α-TOH), gamma-tocopherol (γ-TOH), alpha-tocotrienol (α-TOT), gamma-tocotrienol (γ-TOT), alpha-carboxyethylhydroxychroman (α-CEHC), and gamma-carboxyethylhydroxychroman (γ-CEHC).
   22. The method according to Aspects 20 or 21, wherein assessing the panel of alpha-tocopherol and metabolite levels comprises liquid chromatography-mass spectrometry.
   23. The method according to any of the preceding aspects, wherein the administering is oral administration.
   24. The method according to any of the preceding aspects, wherein the administering is a single administration.
   25. The method according to any of the preceding aspects, wherein the bioavailable alpha-tocopherol is RRR-alpha-tocopherol.
   26. The method according to any of the preceding aspects, wherein the bioavailable alpha tocopherol is administered at in range from 2500 to 10,000 IU/450 kg.
   27. The method according to Aspect 26, wherein the bioavailable alpha tocopherol is administered at in range from 4000 to 6000 IU/450 kg.
   28. A method of detecting a presence or absence of a neuroaxonal dystrophy associated with vitamin E deficiency in a non-human subject, the method comprising:

obtaining a baseline alpha-carboxymethylbutyl hydroxychroman (alpha-CMBHC) concentration for the subject;

administering to the subject a bioavailable alpha-tocopherol;

measuring a post-administration alpha-CMBHC concentration for the subject; and

detecting:
i) the presence of neuroaxonal dystrophy associated with vitamin E deficiency in the subject when the post-administration alpha-CMBHC concentration is increased at least 5-fold as compared to the baseline alpha-CMBHC concentration; or
ii) the absence of neuroaxonal dystrophy associated with vitamin E deficiency in the subject when the post-administration alpha-CMBHC concentration is increased less than 5-fold as compared to the baseline alpha-CMBHC concentration.
   29. The method according to Aspect 28, where the post-administration alpha-CMBHC concentration is increased at least at least a 9-fold.
   30. The method according to Aspect 28 or 29, wherein the non-human subject is a horse.
   31. The method according to Aspect 30, wherein the neuroaxonal dystrophy associated with vitamin E deficiency is equine neuroaxonal dystrophy (eNAD)/equine degenerative myeloencephalopathy (EDM) (eNAD/EDM).
   32. The method according to Aspect 30 or 31, wherein the horse is a quarter horse, a paint/appaloosa, a haflinger, a standardbred, a thoroughbred, a pony, a lusitano/andalusian, a morgan, a paso fino, an arabian, a tennessee walking horse, a norwegian fjord, or a mixed breed.
   33. The method according to any of Aspects 28 to 32, wherein the sample is collected within 24 hours of the administering.
   34. The method according to any of Aspects 28 to 32, wherein the sample comprises serum.
   35. The method according to any of Aspects 28 to 32, wherein the sample comprises plasma.
   36. The method according to any of Aspects 28 to 33, wherein the sample comprises urine.
   37. The method according to any of Aspects 28 to 36, further comprising measuring a pre-administration alpha-CMBHC concentration in a sample from the subject.
   38. The method according to Aspect 37, wherein the method further comprises comparing the post-administration alpha-CMBHC concentration to the pre-administration alpha-CMBHC concentration
   39. The method according to any of Aspects 28 to 38, further comprising measuring a pre-administration alpha-tocopherol concentration in a sample from the subject.
   40. The method according to Aspect 39, further comprising measuring a post-administration alpha-tocopherol concentration in a sample from the subject.
   41. The method according to Aspect 40, wherein the method further comprises comparing the post-administration alpha-tocopherol concentration to the pre-administration alpha-tocopherol concentration.
   42. The method according to any of Aspects 28 to 41, wherein the method comprises measuring a plurality of post-administration alpha-CMBHC concentrations each at a different timepoint following the administration.
   43. The method according to Aspect 42, wherein the method comprises measuring at least a 6 hour post-administration timepoint and a 12 hour post-administration timepoint.
   44. The method according to any of Aspects 28 to 43, wherein the method comprises measuring a plurality of post-administration alpha-tocopherol concentrations each at a different timepoint following the administration.
   45. The method according to Aspect 44, wherein the method comprises measuring at least a 6 hour post-administration timepoint and a 12 hour post-administration timepoint.
   46. The method according to any of Aspects 28 to 45, wherein the measuring comprises liquid chromatography-mass spectrometry.
   47. The method according to any of Aspects 28 to 46, wherein the method comprises assessing a panel of alpha-tocopherol and metabolite levels.
   48. The method according to Aspect 47, wherein the panel of alpha-tocopherol and metabolite levels comprises alpha-CMBHC and one or more of alpha-tocopherol (α-TOH), gamma-tocopherol (γ-TOH), alpha-tocotrienol (α-TOT), gamma-tocotrienol (γ-TOT), alpha-carboxyethylhydroxychroman (α-CEHC), and gamma-carboxyethylhydroxychroman (γ-CEHC).
   49. The method according to Aspects 47 or 48, wherein assessing the panel of alpha-tocopherol and metabolite levels comprises liquid chromatography-mass spectrometry.
   50. The method according to any of Aspects 28 to 49, wherein the administering is oral administration.
   51. The method according to any of Aspects 28 to 50, wherein the administering is a single administration.

52. The method according to any of Aspects 28 to 51, wherein the bioavailable alpha-tocopherol is RRR-alpha-tocopherol.

53. The method according to any of Aspects 28 to 52, wherein the bioavailable alpha tocopherol is administered at in range from 2500 to 10,000 IU/450 kg.
54. The method according to Aspect 53, wherein the bioavailable alpha tocopherol is administered at in range from 4000 to 6000 IU/450 kg.
55. A method of treating a non-human subject for a neuroaxonal dystrophy associated with vitamin E deficiency disorder, the method comprising:

detecting a neuroaxonal dystrophy associated with vitamin E deficiency disorder in the subject according to any of the preceding aspects; and

administering high dose vitamin E to the subject having the neuroaxonal dystrophy associated with vitamin E deficiency disorder.

56. The method according to Aspect 55, wherein the subject is a horse and the high dose vitamin E comprise at least 4.5 IU/kg/day.
57. The method according to Aspect 55 or 56, wherein the subject is a horse and the neuroaxonal dystrophy associated with vitamin E deficiency is equine neuroaxonal dystrophy (eNAD)/equine degenerative myeloencephalopathy (EDM) (eNAD/EDM).
58. The method according to Aspect 55 or 56, wherein the horse is 2 years old or less.
59. A method of screening a non-human subject for breeding, the method comprising:

detecting an absence of a neuroaxonal dystrophy associated with vitamin E deficiency disorder in the subject according to any of Aspects 1 to 54; and

breeding the subject with the detected absence of the neuroaxonal dystrophy associated with vitamin E deficiency disorder.

60. The method according to Aspect 59, wherein the breading comprises artificial insemination.
61. The method according to Aspect 60, wherein the method further comprises collecting a semen sample from the subject with the detected absence of the neuroaxonal dystrophy associated with vitamin E deficiency disorder.
62. The method according to Aspects 60, wherein the method further comprises collecting one or more ova from the subject with the detected absence of the neuroaxonal dystrophy associated with vitamin E deficiency disorder.
63. The method according to Aspect 59, wherein the subject is a horse and the breeding comprises live cover.
64. The method according to Aspect 59, wherein the subject is a horse and the breading comprises one or more advanced reproductive techniques.
65. The method according to Aspect 64, wherein the one or more advanced reproductive techniques are selected from the group consisting of: embryo transfer, gamete intrafallopian transfer (GIFT), egg transfer and intracytoplasmic sperm injection (ICSI).
66. A kit for detecting a neuroaxonal dystrophy associated with vitamin E deficiency disorder, the kit comprising:

a dose of a bioavailable alpha-tocopherol; and

a sample collection container.

67. The kit according to Aspect 66, wherein the neuroaxonal dystrophy associated with vitamin E deficiency is equine neuroaxonal dystrophy (eNAD)/equine degenerative myeloencephalopathy (EDM) (eNAD/EDM).
68. The kit according to Aspect 66 or 67, wherein the dose is formulated for oral delivery as a single dose.
69. The kit according to Aspect 66 or 67, wherein the dose is formulated for injection as a single dose.
70. The kit according to Aspect 68 or 69, wherein the single dose comprises 2500 to 10,000 IU of the bioavailable alpha-tocopherol.
71. The kit according to Aspect 70, wherein the single dose comprises 4000 to 6000 IU of the bioavailable alpha-tocopherol.
72. The kit according to any of Aspects 66 to 71, wherein the kit comprises at least two sample collection containers.

73. A method of detecting a presence or absence of a neuroaxonal dystrophy associated with vitamin E deficiency in a non-human subject, the method comprising:

administering to the subject a bioavailable alpha-tocopherol;

measuring a post-administration alpha-carboxymethylbutyl hydroxychroman (alpha-CMBHC) concentration in a sample from the subject; and

detecting the presence or absence of neuroaxonal dystrophy associated with vitamin E in the subject based on the measured post-administration alpha-CMBHC concentration in the sample.

74. The method according to Aspect 73, wherein:
i) the presence of neuroaxonal dystrophy associated with vitamin E deficiency in the subject is detected when the post-administration alpha-CMBHC concentration is above a predetermined threshold; or
ii) the absence of neuroaxonal dystrophy associated with vitamin E deficiency in the subject is detected when the post-administration alpha-CMBHC concentration is below a predetermined threshold.
   75. The method according to Aspect 73, wherein the method further comprises obtaining a baseline alpha-CMBHC concentration for the subject prior to the administering, and detecting:
i) the presence of neuroaxonal dystrophy associated with vitamin E deficiency in the subject when the post-administration alpha-CMBHC concentration is increased at least 5-fold as compared to the baseline alpha-CMBHC concentration; or
ii) the absence of neuroaxonal dystrophy associated with vitamin E deficiency in the subject when the post-administration alpha-CMBHC concentration is increased less than 5-fold as compared to the baseline alpha-CMBHC concentration.
   76. The method according to any of Aspects 73 to 75, wherein the non-human subject is a horse, optionally wherein the horse is a quarter horse, a paint/appaloosa, a haflinger, a standardbred, a thoroughbred, a pony, a lusitano/andalusian, a morgan, a paso fino, an arabian, a tennessee walking horse, a norwegian fjord, or a mixed breed.
   77. The method according to Aspect 76, wherein the neuroaxonal dystrophy associated with vitamin E deficiency is equine neuroaxonal dystrophy (eNAD)/equine degenerative myeloencephalopathy (EDM) (eNAD/EDM).
   78. The method according to any of Aspects 73 to 77, wherein the sample comprises serum, plasma, or urine.
   79. The method according to any of Aspects 73 to 78, wherein the method comprises measuring a plurality of post-administration alpha-CMBHC concentrations each at a different timepoint following the administration, optionally wherein the method comprises measuring at least a 6 hour post-administration timepoint and a 12 hour post-administration timepoint.
   80. The method according to any of Aspects 73 to 79, wherein the method comprises assessing a panel of alpha-tocopherol and metabolite levels, optionally wherein the panel of alpha-tocopherol and metabolite levels comprises alpha-CMBHC and one or more of alpha-tocopherol (α-TOH), gamma-tocopherol (γ-TOH), alpha-tocotrienol (α-TOT), gamma-tocotrienol (γ-TOT), alpha-carboxyethylhydroxychroman (α-CEHC), and gamma-carboxyethylhydroxychroman (γ-CEHC).
   81. A method of treating a non-human subject for a neuroaxonal dystrophy associated with vitamin E deficiency disorder, the method comprising:

a) detecting, or having detected, a neuroaxonal dystrophy associated with vitamin E deficiency disorder in the subject according to any of Aspects 73 to 80; and

b) administering high dose vitamin E to the subject having the neuroaxonal dystrophy associated with vitamin E deficiency disorder.

82. The method according to Aspect 81, wherein the subject is a horse and the high dose vitamin E comprise at least 4.5 IU/kg/day.
83. The method according to Aspect 82, wherein the horse is 2 years old or less.
84. A method of screening a non-human subject for breeding, the method comprising:

a) detecting an absence of a neuroaxonal dystrophy associated with vitamin E deficiency disorder in the subject according to any of Aspects 73 to 80; and

b) breeding the subject with the detected absence of the neuroaxonal dystrophy associated with vitamin E deficiency disorder.

85. The method according to Aspect 84, wherein the breading comprises artificial insemination, collecting a semen sample or one or more ova from the subject, one or more advanced reproductive techniques, or a combination thereof.
86. The method according to Aspect 85, wherein the one or more advanced reproductive techniques are selected from the group consisting of: embryo transfer, gamete intrafallopian transfer (GIFT), egg transfer, and intracytoplasmic sperm injection (ICSI).
87. A kit for detecting a neuroaxonal dystrophy associated with vitamin E deficiency disorder, the kit comprising:

a dose of a bioavailable alpha-tocopherol; and

a sample collection container.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of detecting a presence or absence of a neuroaxonal dystrophy associated with vitamin E deficiency in a non-human subject, the method comprising:
administering to the subject a bioavailable alpha-tocopherol;
measuring a post-administration alpha-carboxymethylbutyl hydroxychroman (alpha-CMBHC) concentration in a sample from the subject; and
detecting the presence or absence of neuroaxonal dystrophy associated with vitamin E in the subject based on the measured post-administration alpha-CMBHC concentration in the sample.

2. The method according to claim 1, wherein:
i) the presence of neuroaxonal dystrophy associated with vitamin E deficiency in the subject is detected when the post-administration alpha-CMBHC concentration is above a predetermined threshold; or
ii) the absence of neuroaxonal dystrophy associated with vitamin E deficiency in the subject is detected when the post-administration alpha-CMBHC concentration is below a predetermined threshold.

3. The method according to claim 1, wherein the method further comprises obtaining a baseline alpha-CMBHC concentration for the subject prior to the administering, and detecting:
i) the presence of neuroaxonal dystrophy associated with vitamin E deficiency in the subject when the post-administration alpha-CMBHC concentration is increased at least 5-fold as compared to the baseline alpha-CMBHC concentration; or
ii) the absence of neuroaxonal dystrophy associated with vitamin E deficiency in the subject when the post-administration alpha-CMBHC concentration is increased less than 5-fold as compared to the baseline alpha-CMBHC concentration.

4. The method according to any of the preceding claims, wherein the non-human subject is a horse, optionally wherein the horse is a quarter horse, a paint/appaloosa, a haflinger, a standardbred, a thoroughbred, a pony, a lusitano/andalusian, a morgan, a paso fino, an arabian, a tennessee walking horse, a norwegian fjord, or a mixed breed.

5. The method according to claim 4, wherein the neuroaxonal dystrophy associated with vitamin E deficiency is equine neuroaxonal dystrophy (eNAD)/equine degenerative myeloencephalopathy (EDM) (eNAD/EDM).

6. The method according to any of the preceding claims, wherein the sample comprises serum, plasma, or urine.

7. The method according to any of the preceding claims, wherein the method comprises measuring a plurality of post-administration alpha-CMBHC concentrations each at a different timepoint following the administration, optionally wherein the method comprises measuring at least a 6 hour post-administration timepoint and a 12 hour post-administration timepoint.

8. The method according to any of the preceding claims, wherein the method comprises assessing a panel of alpha-tocopherol and metabolite levels, optionally wherein the panel of alpha-tocopherol and metabolite levels comprises alpha-CMBHC and one or more of alpha-tocopherol (α-TOH), gamma-tocopherol (γ-TOH), alpha-tocotrienol (α-TOT), gamma-tocotrienol (γ-TOT), alpha-carboxyethylhydroxychroman (α-CEHC), and gamma-carboxyethylhydroxychroman (γ-CEHC).

9. A method of treating a non-human subject for a neuroaxonal dystrophy associated with vitamin E deficiency disorder, the method comprising:
a) detecting, or having detected, a neuroaxonal dystrophy associated with vitamin E deficiency disorder in the subject according to any of the preceding claims; and
b) administering high dose vitamin E to the subject having the neuroaxonal dystrophy associated with vitamin E deficiency disorder.

10. The method according to claim 9, wherein the subject is a horse and the high dose vitamin E comprise at least 4.5 IU/kg/day.

11. The method according to claim 10, wherein the horse is 2 years old or less.

12. A method of screening a non-human subject for breeding, the method comprising:
a) detecting an absence of a neuroaxonal dystrophy associated with vitamin E deficiency disorder in the subject according to any of claims 1 to 8; and
b) breeding the subject with the detected absence of the neuroaxonal dystrophy associated with vitamin E deficiency disorder.

13. The method according to claim 12, wherein the breading comprises artificial insemination, collecting a semen sample or one or more ova from the subject, one or more advanced reproductive techniques, or a combination thereof.

14. The method according to claim 13, wherein the one or more advanced reproductive techniques are selected from the group consisting of: embryo transfer, gamete intrafallopian transfer (GIFT), egg transfer, and intracytoplasmic sperm injection (ICSI).

15. A kit for detecting a neuroaxonal dystrophy associated with vitamin E deficiency disorder, the kit comprising:
a dose of a bioavailable alpha-tocopherol; and
a sample collection container.
